(19) United States
(12) Patent Application Publication
(10) Pub. No.: US 2024/0239933 A1
(43) Pub. Date: Jul. 18, 2024

(54) METHOD FOR OBTAINING A BIO-SOURCED-MONOMER FROM RENEWABLE DIMETHYLAMINOETHANOL

(71) Applicant: SNF Group, ANDREZIEUX BOUTHEON (FR)

(72) Inventors: Cédrick FAVERO, ANDREZIEUX BOUTHEON (FR); Johann KIEFFER, ANDREZIEUX BOUTHEON (FR)

(21) Appl. No.: 18/562,722

(22) PCT Filed: Jul. 8, 2022
(86) PCT No.: PCT/EP2022/069136

§ 371 Date: Nov. 20, 2023

(30) Foreign Application Priority Data
Jul. 9, 2021 (FR) 2107495

(51) Int. Cl. C08F 220/34; C02F 1/56; C07C 213/06; C07C 219/08; C09K 8/68; C09K 8/80; C12P 13/00; D21H 17/37; D21H 21/10; D21H 23/14; E21B 43/27

(52) U.S. Cl. CPC C08F 220/34; C02F 1/56; C07C 213/06; C07C 219/08; C09K 8/68; C09K 8/80; C12P 13/001; D21H 17/375; D21H 21/10; D21H 23/14; E21B 43/27

(57) ABSTRACT

A method for obtaining dimethylaminoethyl (meth)acrylate comprising reacting a (meth)acrylic ester with dimethylaminoethanol that-is at least partially renewable and non-fossil. A bio-sourced dimethylaminoethyl (meth)acrylate has a bio-sourced carbon content ranging between 45 wt % and 100 wt % relative to the total carbon weight in the bio-sourced dimethylaminoethyl (meth)acrylate. The bio-sourced carbon content can be measured according to ASTM D6866-21 Method B.

### FIELD OF THE INVENTION

The present invention relates to a method for obtaining a monomer from dimethylaminoethanol that is at least partially renewable and non-fossil, said monomer being preferably dimethylaminoethyl acrylate, dimethylaminoethyl methacrylate, salified versions of same or quaternized versions of same. In a preferred embodiment, said method is a biological method comprising enzymatic hydrolysis of said dimethylaminoethanol in the presence of a biocatalyst comprising a hydrolase enzyme, preferably a lipase enzyme.

The invention equally relates to a bio-sourced polymer obtained from the monomer according to the invention, as well as to the use of said bio-sourced polymers in various technical fields.

### Prior Art

Dimethylaminoethyl acrylate and dimethylaminoethyl methacrylate and their salified or quaternized versions are monomers widely used in making water-soluble polymers.

The reaction implemented in the method for preparing dimethylaminoethyl (meth)acrylate follows the reaction pattern hereinafter, wherein a short-alkyl-chain acrylic ester generally methyl, ethyl or butyl acrylate, reacts with dimethylaminoethanol.

It is known to the person skilled in the art that dimethylaminoethyl (meth)acrylate cannot be obtained in an industrial and viable manner by a direct esterification without a catalyst between acrylic acid (or an acrylic ester) and dimethylaminoethanol. Indeed, without a catalyst, dimethylaminoethanol tends to react on the double bond to form an undesired product which are Michael adducts.

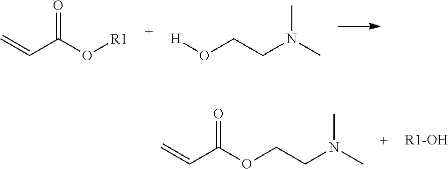

In this reaction, R1 is an alkyl chain of 1 to 4 carbon atoms, linear or branched. Consequently, many patents describe the use of catalysts to increase the conversion and selection of the reaction, in order to minimize the secondary Michael adduct reaction in which dimethaminoethanol adds to the double bond of the acrylic derivative (ester or acid).

For example, Document U.S. Pat. No. 6,437,173 describes the use of titanate catalyst. Other types of organometallic catalysts can be used, such as organotins, dibutyl tin oxide, dilauryl tin oxide or dichloride tin oxide. More particularly, Document EP 2,435,180 describes the transesterification reaction between methyl acrylate and dimethylaminoethanol in the presence of dibutyl tin oxide. Document EP 1 773 748 describes the whole method for obtaining acrylic acid by oxidation of propane or propylene.

These organometallic catalysts lose all their efficiency in the presence of water, and consequently acrylic acid cannot be used. To achieve this, the person skilled in the art uses a short-alkyl-chain acrylic ester, usually methyl, ethyl or butyl acrylate.

The transesterification reaction between a short-alkyl-chain acrylic ester and dimethylaminoethanol generates an alcohol by-product corresponding to the short alkyl chain. For example methanol is produced when methyl acrylate reacts with dimethylaminoethanol.

Document JP 2000072725 describes a route for obtaining dimethylaminoethanol which is the result of the reaction between ethylene oxide and dimethylamine. Ethylene oxide is obtained by oxidation of ethylene.

Fossil-based ethylene contains various impurities, which remain or are transformed in the method for producing dimethylaminoethanol. for example, we can mention the presence of 2-vinyloxyethanol. In the production of dimethylaminoethyl (meth)acrylate, this impurity will react with the alkyl acrylate to form vinyl oxyethyl methacrylate. This impurity is undesirable during the polymerization of dimethylaminoethyl (meth)acrylate, and heavily impacts polymerization methods as well as the final application performances.

Acrylic acid ester is obtained by esterification between acrylic acid and an alcohol, generally catalyzed with an acid such as para toluene sulfonic acid, Nafion® resin, sulfuric acid, methane sulfonic acid as in Document WO 2015/015100 for example.

There are many documents describing how to obtain bio-sourced acrylic acid, such as Document US 2010/0168471, for example, which claims the transformation of glycerol into acrylic acid, Document WO 2012/074818 which claims the fermentation of biomass to obtain a 3-hydroxypropionic acid intermediate, the latter being a chemical precursor of acrylic acid.

Dimethylaminoethyl (meth)acrylate can be quaternized with an alkylating agent, such as with alkyl halides, and more particularly methyl chloride. Methyl chloride is obtained by reaction of hydrochloric acid and methanol, as described in Document U.S. Pat. No. 5,917,099. Methanol is obtained by oxidation of methane with oxygen.

The problem the invention proposes to resolve is to propose a new and improved method for producing ethylenically unsaturated monomers, such as dimethylaminoethyl acrylate and dimethylaminoethyl methacrylate.

### SUMMARY OF THE INVENTION

Quite surprisingly, the Applicant has observed that the use of dimethylaminoethanol that is at least partially renewable and non-fossil, preferably totally renewable origin, in a method for obtaining a monomer of formula (I) and particularly dimethylaminoethyl acrylate (R₂═H) or dimethylaminoethyl methacrylate (R₂═CH₃), helps to improve the quality (purity) of the monomer obtained, and thus to improve their polymerization and the application performances of the polymers.

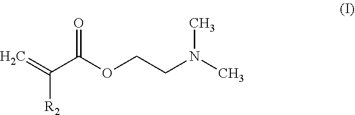

In particular, the Applicant has observed such improvement when the method is a biological method carried out in the presence of a biocatalyst comprising a hydrolase type enzyme, particularly a lipase type enzyme. In this case, it also significantly reduces the consumption of biocatalyst and increases the recycling rate of said biocatalyst.

Without seeking to be bound by any particular theory, the Applicant raises the possibility that the different nature of the impurities between fossil-based dimethylaminoethanol and renewable and non-fossil-based dimethylaminoethanol is the cause of these unexpected technical effects.

The invention firstly relates to a method for obtaining a monomer of formula (I) comprising the reaction between a compound of formula (II) and dimethylaminoethanol, R₂ being a hydrogen atom or a CH₃ group, R₃ being a hydrogen atom or an alkyl group comprising from 1 to 8 carbon atoms, characterized in that the dimethylaminoethanol is at least partially renewable and non-fossil.

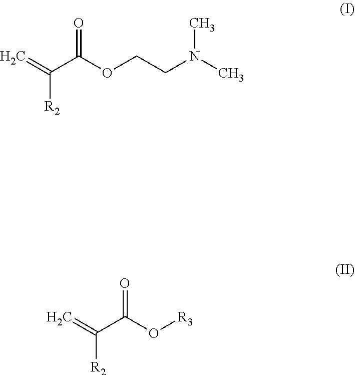

Preferably, R₃ is an alkyl group comprising 1 to 4 carbon atom, and more preferably 1 or 2 carbon atoms.

Dimethylaminoethanol preferably has a bio-sourced carbon content ranging between 5 wt % and 100 wt % relative to the total carbon weight in said dimethylaminoethanol, the bio-sourced carbon content being measured according to ASTM D6866-21 Method B.

In the present description, the expressions “ranging between X and Y” and “from X to Y” include the terminals X and Y.

Polymer or (co)polymer is understood to mean a homopolymer of the monomer of formula (I) or a copolymer of the monomer of formula (I) and at least one monomer different from the monomer of formula (I), for example a terpolymer.

The compound of formula (II) is either an acrylic ester where R₃ is an alkyl group comprising from 1 to 8 carbon atoms, or acrylic acid where R₃ is a hydrogen atom.

The invention further relates to a monomer of formula (I) with a bio-sourced carbon content ranging between 45 wt % and 100 wt % relative to the total carbon weight in said monomer, the bio-sourced carbon content being measured according to ASTM D6866-21 Method B.

The invention also relates to a polymer obtained by polymerization of at least one monomer of formula (I) obtained according to the method of the invention or as previously described, and the use of said polymers in various technical fields.

With the present invention, it is possible to achieve environmental objectives inherent in new technical innovations. In the present case, the use of a renewable raw material, in this case dimethylaminoethanol, helps to significantly optimize the conversion process and the quality of the monomer obtained. Preferably, the use of dimethylaminoethanol and acrylic ester of formula (II) or acrylic acid that are renewable, combined with a biomethod, helps to further improve the quality of the monomers of formula (I), which offer unexpectedly improved performances.

The Applicant observed that, when dimethylaminoethanol is partially or totally of renewable and non-fossil origin, the conversion of dimethylaminoethanol to compound of formula (I) is improved. The purity of compound of formula (I) is also improved.

The Applicant also observed that, when compound (II) is partially or totally of renewable and non-fossil origin, the formation of compound of formula (I) is improved. The purity of compound of formula (I) is also improved.

The Applicant also observed that the polymers according to the invention have an improved biodegradability profile as compared to polymers that do not contain bio-sourced monomers.

The Applicant also observed that the polymers which are entirely of renewable and non-fossil origin have fewer insolubles.

The Applicant also observed that the polymers according to the invention exhibit improved performance as a retention agent for paper, as compared to polymers that do not contain bio-sourced monomers. They also improve the drainage performance.

The Applicant also observed that the polymers according to the invention affords an improved friction reduction as compared to polymers that do not contain bio-sourced monomers.

### DETAILED DESCRIPTION OF THE INVENTION

In the context of the invention, the terms “renewable and non-fossil” are used to designate the origin of a chemical compound derived from biomass or from synthesis gas (syngas), i.e. resulting from one or more chemical transformations carried out on one or more natural and non-fossil raw materials. The terms “bio-sourced” or “bio-resourced” can also be used to characterize the renewable and non-fossil origin of a chemical compound. The renewable and non-fossil origin of a compound includes renewable and non-fossil raw materials stemming from the circular economy, which have been previously recycled, once or several times, in a biomass material recycling process, such as materials from polymer depolymerization or pyrolysis oil processing.

According to the invention, the “at least partially renewable and non-fossil” quality of a compound means a bio-sourced carbon content preferably between 5 wt % and 100 wt % relative to the total carbon weight of said compound.

In the context of the invention, the ASTM D6866-21 standard Method B is used to characterize the bio-sourced nature of a chemical compound and to determine the bio-sourced carbon content of said compound. The value is expressed as a weight percentage (wt %) of bio-sourced carbon relative to the total carbon weight in said compound.

The ASTM D6866-21 standard is a test method that teaches how to experimentally measure the bio-sourced carbon content of solids, liquids and gaseous samples by radiocarbon analysis.

This standard primarily uses Accelerator Mass Spectrometry (AMS) technology. This technique is used to naturally measure the radionuclides present in a sample, wherein the atoms are ionized, then accelerated to high energies, then separated, and individually counted in Faraday cups. This high-energy separation is extremely effective at filtering out isobaric interference, so that AMS is able to accurately measure abundances of carbon-14 relative to carbon-12 (14C/12C) to an accuracy of 1.10⁻¹⁵.

The ASTM D6866-21 standard Method B uses AMS and IRMS (Isotope Ratio Mass Spectroscopy). The test method allows to directly differentiate contemporary carbon-based carbon atoms from fossil-based carbon atoms. A measure of the carbon-14 to carbon-12 or carbon-14 to carbon-13 content of a product is determined against a modern carbon-based reference material accepted by the radiocarbon dating community such as the NIST's Standard Reference Material (SRM) 4990C (oxalic acid).

The sample preparation method is described in the standard and does not require any special comment as it is a commonly used procedure.

Analysis, interpretation and reporting of results are described below. Isotope ratios of carbon-14 to carbon-12 content or carbon-14 to carbon-13 content are measured using AMS. Isotope ratios of carbon-14 to carbon-12 content or carbon-14 to carbon-13 content are determined relative to a standard traceable via the NIST SRM 4990C modern reference standard. The “fraction of modern” (fM) represents the amount of carbon-14 in the tested product relative to the modern standard. It is most often referred to as percent modern carbon (pMC), the percentage equivalent to fM (e.g. fM 1=100 pMC).

All pMC values obtained from radiocarbon analyses must be corrected for isotopic fractionation using a given stable isotope. The correction should be made using the carbon-14 to carbon-13 values determined directly using the AMS where possible. If this is not possible, the correction should be made using the delta 13C (δ13C) measured by IRMS, CRDS (Cavity Ring Down Spectroscopy) or any other equivalent technology that can provide accuracy to within plus or minus 0.3 per thousand.

“Zero pMC” represents the total absence of measurable 14C in a material above the background signals, thus indicating a fossil (e.g. petroleum-based) carbon source. A value of 100 pMC indicates a fully “modern” carbon source. A pMC value between 0 and 100 indicates a proportion of carbon derived from a fossil source relative to a “modern” source.

The pMC may be higher than 100% due to the persistent, but diminishing, effects of 14C injection into the atmosphere caused by atmospheric nuclear testing programmes. The pMC values need to be adjusted by an atmospheric correction factor (REF) to obtain the actual bio-sourced content of the sample.

The correction factor is based on the excess 14C activity in the atmosphere at the time of testing. A REF value of 102 pMC was determined for 2015 based on CO₂ measurements in the air in a rural area of the Netherlands (Lutjewad, Groningen). The first version of this standard (ASTM D6866-04) in 2004 had referenced a value of 107.5 pMC, while the later version ASTM D6866-10 (2010) had referenced a value of 105 pMC. These data points represent a drop of 0.5 pMC per year. Consequently, on 2 January of each year, the values in Table 1 below were used as REF value until 2019, reflecting the same decrease of 0.5 pMC per year. The REF values (pMC) for 2020 and 2021 have been determined to be 100.0 based on continuous measurements in the Netherlands (Lutjewad, Groningen) until 2019. References for reporting carbon isotope ratio data are provided below for 14C and 13C, respectively Roessler, N., Valenta, R. J., and van Cauter, S., “Time-resolved Liquid Scintillation Counting”, Liquid Scintillation Counting and Organic Scintillators, Ross, H., Noakes, J. E., and Spaulding, J. D., Eds., Lewis Publishers, Chelsea, M I, 1991, pp. 501-511. Allison, C. E., Francy, R. J., and Meijer, H. A. J., “Reference and Intercomparison Materials for Stable Isotopes of Light Elements”, International Atomic Energy Agency, Vienna, Austria, IAEATECHDOC-825, 1995.

The percentage of the bio-sourced carbon content is calculated by dividing pMC by REF and multiplying the result by 100. For example, [102 (pMC)/102 (REF)]×100=100% bio-sourced carbon. The results are indicated as a weight percentage (w %) of bio-sourced carbon relative to the total carbon weight in said compound.

TABLE 1
Reference of percentage of modern carbon (pMC)

| REF year | pMC |
|---|---|
| 2015 | 102.0 |
| 2016 | 101.5 |
| 2017 | 101.0 |
| 2018 | 100.5 |
| 2019 | 100.0 |
| 2020 | 100.0 |
| 2021 | 100.0 |

In the context of the invention, the term “segregated” means a material stream that is distinctive and distinguishable from other material streams in a value chain (e.g. in a product manufacturing method), and thus considered to belong to a set of materials having an equivalent nature, such that the same origin of the material, or its manufacture according to the same standard or norm, can be tracked and guaranteed throughout this value chain.

For example, this may be the case of a chemist buying 100% bio-sourced dimethylaminoethanol from a single supplier who guarantees the 100% bio-sourced origin of the dimethylaminoethanol delivered, and said chemist processing this 100% bio-sourced dimethylaminoethanol separately from other potential dimethylaminoethanol sources to produce a chemical compound. If the chemical compound produced is made solely from said 100% bio-sourced dimethylaminoethanol then the chemical compound is 100% bio-sourced.

In the context of the invention, the term “non-segregated”, in contrast to the term “segregated”, is understood to mean a material stream that cannot be differentiated from other material streams in a value chain.

In order to better understand this notion of segregation, it is useful to recall some basics about the circular economy and its practical application in methods, especially chemical transformation.

According to the French Environment and Energy Management Agency (ADEME), the circular economy can be defined as an economic system of trade and production which, at all stages of the life cycle of products (goods and services), seeks to increase efficiency in the use of resources and to reduce the environmental impact while developing the well-being of individuals. In other words, it is an economic system devoted to efficiency and sustainability that minimizes waste by optimizing value generated by resources. It relies heavily on a variety of conservation and recycling practices in order to break away from the current more linear “take-make-dispose” approach.

In the field of chemistry, which is the science of transforming one substance into another, this translates into reusing material that has already been used to make a product. Theoretically, all chemicals can be isolated and therefore recycled separately from other chemicals. The reality, particularly in industry, is more complex and means that even when isolated, the compound cannot often be differentiated from the same compound originating from another source, thus complicating the traceability of the recycled material.

For this reason, various traceability models have been developed taking into account this industrial reality, thereby allowing users in the chemical industry to manage their material streams with full knowledge of the facts, and allowing end customers to understand and know in a simple way the origin of the materials used to produce an object or a commodity.

These models have been developed to build transparency and trust throughout the value chain. Ultimately, this allows end-users or customers to choose a more sustainable solution without having the ability themselves to control every aspect of the method, by knowing the proportion of a desired component (e.g. of a bio-sourced nature) in an object or commodity.

One such model is “segregation”, which we have defined earlier. Some known examples where this model applies are glass and some metals where it is possible to track material streams separately.

However, chemicals are often used in complex combinations, and separate cycles are very often difficult to implement, especially due to prohibitive costs and highly complex stream management, such that the “segregation” model is not always applicable.

Consequently, when it is not possible to differentiate between material streams, other models are applied, which are grouped together under the term “non-segregated” and which entail, for example, taking into account the proportion of a specific stream relative to other streams, without physically separating the streams. One example is the Mass Balance Approach.

The Mass Balance Approach involves accurately tracking the proportion of a category (e.g. “recycled”) relative to a whole in a production system in order to guarantee, on the basis of an auditable account ledger, a proportionate and appropriate allocation of the content of that category in a finished product.

For example, this may be the case of a chemist buying 50% bio-sourced dimethylaminoethanol from a supplier who guarantees, according to the mass or weight balance approach, that in the dimethylaminoethanol delivered, 50% of the dimethylaminoethanol has a bio-sourced origin, and de facto 50% is not of bio-sourced origin, and the use by said chemist of this 50% bio-sourced dimethylaminoethanol with another stream of 0% bio-sourced dimethylaminoethanol, the two streams not being identifiable at some point during the production process, due to mixing for example. If the chemical compound produced is made from 50% bio-sourced 50 wt % guaranteed dimethylaminoethanol, and 0% bio-sourced 50 wt % dimethylaminoethanol, the chemical compound is 25% bio-sourced.

In order to guarantee the stated “bio-sourced” figures, for example, and to encourage the use of recycled raw materials in producing new products, a set of globally shared and standardised rules (ISCC+, ISO 14020) has been developed to reliably manage material streams.

In the context of the invention, the term “recycled” is understood to mean the origin of a chemical compound derived from a method for recycling a material considered as waste, i.e. resulting from one or more transformations carried out using at least one recycling method on at least one material generally considered as waste.

The term “water-soluble polymer” is understood to mean a polymer which gives a clear aqueous solution when dissolved by stirring at 25° C. and with a concentration of 20 g·L⁻¹ in water.

### Method According to the Invention

The present invention therefore relates to a method for obtaining a monomer of formula (I) comprising the reaction between a compound of formula (II) and dimethylaminoethanol, R₂ being a hydrogen atom or a CH₃ group, R₃ being a hydrogen atom or an alkyl group comprising from 1 to 8 carbon atoms, characterized in that the dimethylaminoethanol is at least partially renewable and non-fossil.

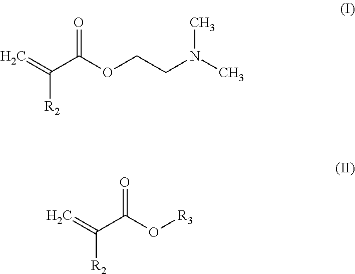

R₃ is preferably an alkyl group comprising 1 to 4 carbon atom, and more preferably 1 or 2 carbon atoms.

Dimethylaminoethanol preferably has a bio-sourced carbon content ranging between 5 wt % and 100 wt % relative to the total carbon weight in said dimethylaminoethanol, the bio-sourced carbon content being measured according to ASTM D6866-21 Method B.

In the whole invention, the bio-sourced carbon content of a compound for which it is specified that it is at least partially renewable and non-fossil, or for which the bio-sourced carbon content is specified, relative to the total carbon weight in said compound, ranges from 5 wt % to 100 wt %, and preferably from 10 wt % to 100 wt %, preferably from 15 wt % to 100 wt %, preferably from 20 wt % to 100 wt %, preferably from 25 wt % to 100 wt %, preferably from 30 wt % to 100 wt %, preferably from 35 wt % to 100 wt %, preferably from 40 wt % to 100 wt %, preferably from 45 wt % to 100 wt %, preferably from 50 wt % to 100 wt %, preferably from 55 wt % to 100 wt %, preferably from 60 wt % to 100 wt %, preferably from 65 wt % to 100 wt %, preferably from 70 wt % to 100 wt %, preferably from 75 wt % to 100 wt %, preferably from 80 wt % to 100 wt %, preferably from 85 wt % to 100 wt %, preferably from 90 wt % to 100 wt %, preferably from 95 wt % to 100 wt %, preferably from 97 wt % to 100 wt %, preferably from 99 wt % to 100 wt %, the bio-sourced carbon content being measured according to ASTM D6866-21 Method B.

As concerns the formula (I) monomer according to the invention, the bio-sourced carbon content relative to the total carbon weight in said monomer, preferentially ranges between 45 wt % and 100 wt %, preferably between 50 wt % and 100 wt %, preferably between 55 wt % and 100 wt %, preferably between 60 wt % and 100 wt %, preferably between 65 wt % and 100 wt %, preferably between 70 wt % and 100 wt %, preferably between 75 wt % and 100 wt %, preferably between 80 wt % and 100 wt %, preferably between 85 wt % and 100 wt %, preferably between 90 wt % and 100 wt %, preferably between 95 wt % and 100 wt %, preferably between 97 wt % and 100 wt %, preferably between 99 wt % and 100 wt %, the bio-sourced carbon content being measured according to the standard ASTM D6866-21 Method B.

Preferentially, the formula (II) compound is at least renewable and non-fossil. Preferentially, it has a bio-sourced carbon content ranging between 25 wt % and 100 wt %, preferably 50 wt % and 100 wt %, more preferably between 75 wt % and 100 wt %, based on the total carbon weight said of formula (II) acrylic ester or acrylic acid, the bio-sourced carbon content being measured according to ASTM D6866-21 Method B.

Preferably, the dimethylaminoethanol is totally renewable and non-fossil. Preferably, the formula (I) monomer is totally renewable and non-fossil. Preferably, the dimethylaminoethanol and the formula (I) monomer are totally renewable and non-fossil.

The formula (I) monomer is dimethylaminoethyl acrylate or dimethylaminoethyl methacrylate.

In a particular embodiment, the formula (I) monomer is either salified or quaternized with an alkylating agent, preferentially with an alkyl halide, e.g. methyl chloride, or dialkyl sulfate, e.g. dimethyl sulfate, diethyl sulfate, or benzyl chloride. The preferred alkylating agent is methyl chloride.

Preferably, the alkylating agent has a bio-sourced carbon content ranging between 50 wt % and 100 wt %, preferably between 70 wt % and 100 wt %, even more preferably of 100 wt % relative to the total carbon weight in said alkylating agent, the bio-sourced carbon content being measured according to ASTM D6866-21 Method B.

Preferably, the dimethylaminoethanol, the formula (I) monomer and the alkylating agent are totally renewable and non-fossil.

In a particularly preferred embodiment, the method is a biological method carried out in the presence of a biocatalyst comprising a hydrolase enzyme selected from lipases, esterases, glycosylases, proteases in free form or immobilized on a substrate. Preferentially, it is preferably a lipase or an esterase.

Preferably, the enzyme is a lipase synthesized by a microorganism advantageously selected from Alcaligenes sp., Aspergillus sp., Mucor sp., Penicillium sp., Geotrichum sp., Rhizopus sp., Burkholderia sp., Candida sp., Pseudomonas sp., Thermomyces sp., Candida Antarctica. Preferentially, the lipase is derived from a Candida Antartica type microorganism.

The dimethylaminoethanol, and/or the formula (II) compound, and/or the alkylating agent may be non-segregated, partially segregated, or totally segregated.

Where the dimethylaminoethanol, and/or the formula (II) compound, and/or the alkylating agent is totally renewable and non-fossil, it may be either:
a) Totally of recycled origin and
a)1) Or totally segregated;
a)2) Or partially segregated;
a)3) Or non-segregated;
b) Or partially of recycled origin and
b)1) Or totally segregated;
b)2) Or partially segregated;
b)3) Or non-segregated;
c) Or totally of non-recycled origin and
c)1) Or totally segregated;
c)2) Or partially segregated;
c)3) Or non-segregated.

In these various embodiments, where the dimethylaminoethanol, and/or the formula (II) compound, and/or the alkylating agent is partially segregated, the weight ratio between the “segregated” part and the “non-segregated” part is preferably between 99:1 and 10:90, preferably between 99:1 and 30:70, or more preferably between 99:1 and 50:50.

Among these various embodiments, preference is given to the three a) embodiments, the three b) embodiments, and embodiment c)1). Among these embodiments, much greater preference is given to embodiments a)1), a)2), b)1), b)2) and c)1). The two most preferred embodiments are a)1) and b)1).

The industrial reality is such that it is not always possible to obtain industrial quantities of dimethylaminoethanol, and/or the formula (II) compound, and/or the alkylating agent that are bio-sourced, totally recycled and/or segregated or highly recycled and segregated. Hence, the above preferences may be more difficult to implement at the moment. From a practical standpoint, embodiments a)3), b)3), and c) are currently implemented more easily and on a larger scale. With techniques evolving quickly towards the circular economy, there is no doubt that the already applicable preferred modes will soon be applicable on a very large scale.

Where the dimethylaminoethanol, and/or the formula (II) compound, and/or the alkylating agent partially renewable and non-fossil, a distinction is made between the renewable part (bio-sourced) and the non-bio-sourced part. Obviously, each of these parts can be according to the same embodiments a), b) and c) described hereinabove.

As concerns the bio-sourced part of the partially bio-sourced dimethylaminoethanol, and/or the formula (II) compound, and/or the alkylating agent, the same preferences apply as in the case where the compound is fully bio-sourced.

However, as concerns the non-bio-sourced part of the partially bio-sourced compound, it is even more preferable to have as large a recycled component as possible for a circular economy approach. Hence, in this case, preference is given to embodiments a)1), a)2), b)1), b)2), particularly a)1) and b)1).

Compound (II) can be obtained by reaction between acrylic acid and an alcohol having an alkyl chain comprising between 1 and 8 carbons. The reaction can be conducted in a batch, semi-batch or continuous manner. The molar ratio between the alcohol and the acrylic acid ranges between 1 and 10, preferably between 1 and 5, even more preferably between 1 and 2.

When the reaction (acrylic acid/acrylic ester—alcohol) is enzymatic, it is generally carried out at a temperature ranging between 5 and 35° C., generally at room temperature. Where the reaction is not enzymatic, it is generally conducted at a temperature between 30 and 150° C., preferably between 50 and 120° C.

The reaction time (acrylic acid/acrylic ester—alcohol) is generally between 1 minute and 24 hours.

The reaction (acrylic acid/acrylic ester—alcohol) can be carried out in the presence of an acid or base catalyst. It can be homogeneous or heterogeneous.

Dimethylaminoethanol can be obtained by reaction between dimethylamine and ethylene oxide. The reaction can be conducted in a batch, semi-batch or continuous manner. Preferentially, the dimethylamine is introduced first into the synthesis reactor, and then ethylene oxide is added. The molar ratio between dimethylamine and ethylene oxide is generally between 1 and 10, preferably between 1 and 5.

The reaction (dimethylamine—ethylene oxide) is generally conducted at a temperature between 50 and 200° C., preferably between 70 and 180C. The reaction time is generally between 1 minute and 24 hours.

The alkylating agent, can be obtained by reaction between an alcohol and a BrØnsted acid, e.g., methanol and hydrochloric acid to form methyl chloride. The reaction can be conducted in a batch, semi-batch or continuous manner. The molar ratio between the alcohol and the BrØnsted acid generally ranges between 1 and 10, preferably between 1 and 5. The BrØnsted acid, for example hydrochloric acid, can be in liquid or gaseous form. Preferably, it is in gaseous form. It can also be in anhydrous form or in aqueous solution. Preferably, it is in anhydrous form.

The reaction (formation of the alkylating agent) is generally carried out at a temperature between 30 and 150° C., preferably between 50 and 120° C. The reaction time is generally between 1 minute and 24 hours. The reaction can be conducted in the presence of an acid or base catalyst or a metal salt. It can be homogeneous or heterogeneous.

The alkylating agent can also be obtained by halogenation of an alkane, for example by chlorination of methane with chlorine gas, at a temperature generally ranging between 400 and 500° C.

In a particular embodiment applicable to the various processes described in the invention, the dimethylaminoethanol, and/or the formula (II) compound, and/or the alkylating agent are partially or totally derived from a recycling process.

The recycling method, may be polymer depolymerization or synthesis from pyrolysis oil, the latter generally resulting from high-temperature, anaerobic combustion of used plastic waste. Thus, materials considered as waste can be used as a source to produce recycled compounds, which in turn can be used as raw material to manufacture the invention's monomer. Since the monomer according to the invention is derived using a recycling method, the polymer according to the invention hereinafter described can cater to the virtuous circle of the circular economy.

In this particular embodiment of the invention, the method for preparing the formula (I) compound according to the invention comprises the following steps:
Recycling at least one material that is at least partially renewable and non-fossil to obtain dimethylaminoethanol, and/or the formula (II) compound;
Reacting the dimethylaminoethanol and the formula (II) compound to obtain the formula (I) compound, preferably according to a biological method conducted in the presence of a biocatalyst comprising a hydrolase enzyme,
Optionally, reacting the formula (I) compound with an alkylating agent.

In another particular embodiment of the invention, the method for preparing the formula (I) compound according to the invention comprises the following steps:
Reacting dimethylaminoethanol and the formula (II) compound to obtain the formula (I) compound, preferably according to a biological method carried out in the presence of a biocatalyst comprising a hydrolase enzyme; the dimethylaminoethanol, and/or the formula (II) compound being able to be derived, independently of each other, from material that is at least partially renewable and non-fossil,
Reacting the formula (I) compound with an alkylating agent derived at least from a partially renewable and non-fossil material.

The recycling rate is the weight ratio of the recycled material to the total material.

In this particular embodiment, the part obtained from recycling is preferably totally “segregated”, i.e. is obtained from a separate pipeline and is treated in a separate manner. In an alternative embodiment, it is partially “segregated” and partially “non-segregated”. In this case, the weight ratio between the “segregated” part and the “non-segregated” part is preferably between 99:1 and 10:90, preferably between 99:1 and 30:70, or more preferably between 99:1 and 50:50.

In a particularly preferred embodiment, the biological method for bioconverting a formula (II) compound and dimethylaminoethanol to obtain the formula (I) monomer comprises enzymatic hydrolysis in the presence of a biocatalyst comprising an enzyme. The bioconversion may be carried out in an aqueous medium, using water in this case as solvent and reagent. As concerns the steps and conditions of the method, the person skilled in the art may refer to his/her general knowledge.

### Monomer According to the Invention

The invention further relates to a formula (I) monomer with a bio-sourced carbon content ranging between 45 wt % and 100 wt %, preferably between 70 wt % and 100 wt %, relative to the total carbon weight in said monomer, the bio-sourced carbon content being measured according to ASTM D6866-21 Method B.

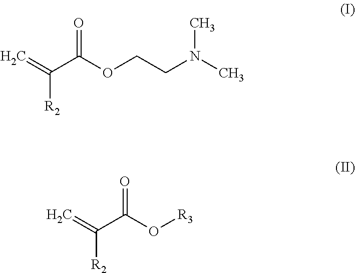

The preferences developed in the method section apply to this section describing the monomer.

The invention further relates to a formula (I) monomer obtained by reacting a formula (II) compound, R₂ being a hydrogen atom or a CH₃ group, R₃ being a hydrogen atom or an alkyl group comprising from 1 to 8 carbon atoms, with dimethylaminoethanol, preferably by a biological process carried out in the presence of a biocatalyst comprising a hydrolase enzyme said dimethylaminoethanol having a bio-sourced carbon content of between 5 wt % and 100 wt % based on the total weight of carbon in said dimethylaminoethanol, and/or, preferably, said compound of formula (II) having a bio-sourced carbon content of between 5 wt % and 100 wt % based on the total weight of carbon in said compound of formula (II), the bio-sourced carbon content being measured according to ASTM D6866-21 Method B.

Preferably, the dimethylaminoethanol is totally renewable and non-fossil. Preferably, the formula (II) compound is totally renewable and non-fossil. Preferably, the formula (I) monomer is partially, preferentially totally renewable and non-fossil. Preferably, the dimethylaminoethanol, the formula (II) compound and the formula (I) monomer are totally renewable and non-fossil.

The invention further relates to a bio-sourced-dimethylaminoethyl (meth)acrylate with a bio-sourced carbon content ranging between 45 wt % and 100 wt % relative to the total carbon weight in said bio-sourced-dimethylaminoethyl (meth)acrylate, the bio-sourced carbon content being measured according to ASTM D6866-21 Method B.

The term “(meth)acrylate” refers to methacrylate or acrylate.

Bio-sourced-dimethylaminoethyl (meth)acrylate is understood to mean a dimethylaminoethyl (meth)acrylate acrylamide that is at least partially, preferably totally derived from biomass, i.e. being the result of one or more chemical transformations carried out on one or more raw materials having a natural, and as opposed to non-fossil, origin. Bio-sourced-dimethylaminoethyl (meth)acrylate can also be called bio-sourced or bio-resourced dimethylaminoethyl (meth)acrylate.

The invention relates to a bio-sourced-dimethylaminoethyl (meth)acrylate obtained by reacting methyl (meth)acrylate with a dimethylaminoethanol, preferably by a biological method conducted in the presence of a biocatalyst comprising a hydrolase enzyme. In this reaction, said dimethylaminoethanol and/or said methyl (meth)acrylate have a bio-sourced carbon content ranging between 45 wt % and 100 wt % based on the total carbon weight in said dimethylaminoethanol and/or said methyl (meth)acrylate, respectively, the bio-sourced carbon content being measured according to ASTM D6866-21 Method B.

The invention further relates to the salified or quaternized form of bio-sourced-dimethylaminoethyl (meth)acrylate. It may be quaternized with an alkylating agent, preferentially with an alkyl halide, e.g. methyl chloride, or dialkyl sulfate, e.g. dimethyl sulfate, diethyl sulfate, or benzyl chloride. The preferred alkylating agent is methyl chloride.

Preferably, the alkylating agent has a bio-sourced carbon content ranging between 50 wt % and 100 wt %, preferably between 70 wt % and 100 wt %, even more preferably of 100 wt % relative to the total carbon weight in said alkylating agent, the bio-sourced carbon content being measured according to ASTM D6866-21 Method B.

Preferably, the dimethylaminoethanol, the formula (I) monomer and the alkylating agent are totally renewable and non-fossil.

The dimethylaminoethanol, and/or the formula (II) compound, and/or the alkylating agent may be non-segregated, partially segregated, or totally segregated. The preferences developed in the method section apply to this section describing the monomer.

In a particular embodiment, the dimethylaminoethanol, and/or the formula (II) compound, and/or the alkylating agent may be partially or totally recycled. The preferences developed in the method section apply to this section describing the monomer.

### Polymer According to the Invention

The invention further relates to a polymer obtained by polymerization of at least one monomer obtained according to the method according to the invention. It also relates to a polymer obtained by polymerization of at least one monomer as previously described. The preferences developed in the method section apply to this section describing the polymer.

The polymer according to the invention is preferably water-soluble or water-swellable. The polymer may also be a superabsorbent.

The polymer according to the invention may be a homopolymer or a copolymer with at least one first monomer obtained according to the method according to the invention, or with at least one previously described first monomer, and with at least one different second monomer, the latter advantageously being chosen from at least one nonionic monomer, and/or at least one anionic monomer, and/or at least one cationic monomer, and/or at least one zwitterionic monomer, and/or at least one monomer comprising a hydrophobic grouping.

Thus, the copolymer may comprise at least a second monomer different from the first monomer, this second monomer being chosen from nonionic monomers, anionic monomers, cationic monomers, zwitterionic monomers, monomers comprising a hydrophobic grouping, and mixtures thereof.

The nonionic monomer is preferably selected from acrylamide, methacrylamide, N-isopropylacrylamide, N,N-dimethylacrylamide, N,N-diethylacrylamide, N-methylolacrylamide, N-vinylformamide (NVF), N-vinylacetamide, N-vinylpyridine and N-vinylpyrrolidone (NVP), N-vinyl imidazole, N-vinyl succinimide, acryloyl morpholine (ACMO), acryloyl chloride, glycidyl methacrylate, glyceryl methacrylate, and diacetone acrylamide.

The anionic monomer is preferably chosen from acrylic acid, methacrylic acid, itaconic acid, crotonic acid, maleic acid, fumaric acid, acrylamido undecanoic acid, 3-acrylamido 3-methylbutanoic acid, maleic anhydride, 2-acrylamido-2-methylpropane sulfonic acid (ATBS), vinylsulfonic acid, vinylphosphonic acid, allylsulfonic acid, methallylsulfonic acid, 2-sulfoethylmethacrylate, sulfopropylmethacrylate, sulfopropylacrylate, allylphosphonic acid, styrene sulfonic acid, 2-acrylamido-2-methylpropane disulfonic acid, and the water-soluble salts of these monomers, such as their alkali metal, alkaline earth metal or ammonium salts. It is preferably acrylic acid (and/or a salt thereof), and/or ATBS (and/or a salt thereof).

The cationic monomer is preferably chosen from quaternized dimethylaminoethyl acrylate (ADAME), quaternized dimethylaminoethyl methacrylate (MADAME), dimethyldiallylammonium chloride (DADMAC), acrylamido propyltrimethyl ammonium chloride (APTAC), and methacrylamido propyltrimethyl ammonium chloride (MAPTAC).

The zwitterionic monomer can be a derivative of a vinyl-type unit, particularly acrylamide, acrylic, allylic or maleic, this monomer having an amine or ammonium function (advantageously quaternary) and an acid function of the carboxylic (or carboxylate), sulfonic (or sulfonate) or phosphoric (or phosphate) type.

Monomers having a hydrophobic character can also be used in preparation of the polymer. Preferably, they are chosen from the group composed of esters of (meth)acrylic acid having an alkyl, arylalkyl, propoxylated, ethoxylated or ethoxylated and propoxylated chain; derivatives of (meth)acrylamide having an alkyl, arylalkyl, propoxylated, ethoxylated, ethoxylated and propoxylated chain, or dialkyl; alkyl aryl sulfonates, or of mono- or di-substituted amides of (meth)acrylamide having a propoxylated, ethoxylated, or ethoxylated and propoxylated alkyl, arylalkyl chain; derivatives of (meth)acrylamide having a propoxylated, ethoxylated, ethoxylated and propoxylated alkyl, arylalkyl, or dialkyl chain; alkyl aryl sulfonates

Each of these monomers may also be bio-sourced.

According to the invention, the polymer may have a linear, branched, star, comb, dendritic or block structure. These structures can be obtained by selecting the initiator, the transfer agent, the polymerization technique such as controlled radical polymerization referred to as RAFT (reversible addition-fragmentation chain transfer), NMP (Nitroxide Mediated Polymerization) or ATRP (Atom Transfer Radical Polymerization), incorporation of structural monomers, the concentration.

According to the invention, the polymer is advantageously linear and structured. A structured polymer refers to a non-linear polymer with side chains so as to obtain, when this polymer is dissolved in water, a pronounced state of entanglement leading to very substantial low gradient viscosities. The invention's polymer may also be cross-linked.

Additionally, the polymer according to the invention polymer may be structured:
By at least one structuring agent, which may be chosen from the group comprising polyethylenically unsaturated monomers (having at least two unsaturated functions), such as vinyl functions for example, particularly allyl, acrylic and epoxy functions, and one may mention, for example, methylene bis acrylamide (MBA), triallylamine, or tetraalkylammonium chloride or 1,2 di hydroxyethylene bis-(N-acrylamide), and/or
By macroinitiator, such as polyperoxides, polyazoids and polytransfer agents, such as polymeric (co)polymers, and polyols, and/or
Functionalized polysaccharides.

The amount of branching/cross-linking agent in the monomer mixture is advantageously less than 4 wt % relative to the monomer content (weight), more advantageously less than 1%, and even more advantageously less than 0.5%. According to a particular embodiment, it may be at least equal to 0.00001 wt % relative to the monomer content.

In a particular embodiment, the polymer according to the invention may be a semi-synthetic and thus semi-natural polymer. In this embodiment, the polymer may be synthesized by copolymerization by total or partial grafting of at least one monomer according to the invention, and at least one natural compound, said natural compound being preferably chosen from starches and their derivatives, polysaccharides and their derivatives, fibers, vegetable gums, animal gums or algal gums, and modified versions thereof. For example, vegetable gums can include guar gum, gum arabic, locust bean gum, gum tragacanth, guanidinium gum, cyanine gum, tara gum, cassia gum, xanthan gum, ghatti gum, karaya gum, gellan gum, cyamopsis tetragonoloba gum, soy gum, or beta-glucan or dammar. The natural compound can also be gelatin, casein, or chitosan. For example, algal gum can include sodium alginate or its acid, agar-agar, or carrageenan.

Polymerization is generally carried out, without this being limiting, by copolymerization or by grafting. The person skilled in the art will be able to refer to current general knowledge in the field of semi-natural polymers.

The invention also relates to a composition comprising at least one polymer according to the invention and at least one natural polymer, said natural polymer being preferably chosen from the previously described natural polymers. The weight ratio between the synthetic polymer and the natural polymer is generally between 90:10 and 10:90. The composition may be in liquid, inverse emulsion or powder form.

In general, the polymer does not require development of a particular polymerization method. Indeed, it can be obtained according to all the polymerization techniques well known to the person skilled in the art. In particular, it can be solution polymerization; gel polymerization; precipitation polymerization; emulsion polymerization (aqueous or inverse); suspension polymerization; reactive extrusion polymerization; water-in-water polymerization; or micellar polymerization.

Polymerization is generally free radical polymerization preferably by inverse emulsion polymerization or gel polymerization. Free radical polymerization includes free radical polymerization using UV, azo, redox or thermal initiators as well as controlled radical polymerization (CRP) techniques or matrix polymerization techniques.

The polymer according to the invention can be modified after it being obtained by polymerization. This is known as post-modification of the polymer. All known post-modifications can be applied to the polymer according to the invention, and the invention also relates to polymers obtained after said post-modifications. Among the possible post-modifications developed hereinafter, mention may be made of post-hydrolysis, post-modification by Mannich reaction, post-modification by Hoffman reaction and post-modification by glyoxalation reaction.

The polymer according to the invention can be obtained by performing a post-hydrolysis reaction on a polymer obtained by polymerization of at least one monomer obtained by the method according to the invention or at least one monomer as previously described in the “Monomer” section. Prior to post-hydrolysis, the polymer comprises acrylamide or methacrylamide monomer units, for example. The polymer may also further comprise monomeric units of N-Vinylformamide. More specifically, post-hydrolysis involves reaction of hydrolyzable functional groups of advantageously non-ionic monomeric units, more advantageously amide or ester functions, with a hydrolysis agent. This hydrolysis agent may be an enzyme, an ion exchange resin, an alkali metal, or a suitable acid compound. Preferably, the hydrolysis agent is a BrØnsted base. Where the polymer comprises amide and/or ester monomer units, the post-hydrolysis reaction produces carboxylate groups. Where the polymer comprises vinylformamide monomer units, the post-hydrolysis reaction produces amine groups.

The polymer according to the invention can be obtained by performing a Mannich reaction on a polymer obtained by polymerization of at least one monomer obtained by the method according to the invention or at least one monomer as previously described in the “Monomer” section. More specifically, prior to the Mannich reaction, the polymer advantageously comprises acrylamide and/or methacrylamide monomer units. The Mannich reaction is performed in aqueous solution in the presence of a dialkyl amine and a formaldehyde precursor. More advantageously, the dialkyl amine is dimethylamine and the formaldehyde precursor is formaldehyde itself. After this reaction, the polymer contains tertiary amines.

The polymer according to the invention can be obtained by performing a Hoffman reaction on a polymer obtained by polymerization of at least one monomer obtained by the method according to the invention or at least one monomer as previously described in the “Monomer” section. Prior to the Hoffman reaction, the polymer advantageously comprises acrylamide and/or methacrylamide monomer units. The so-called Hofmann degradation reaction is carried out in aqueous solution in the presence of an alkaline earth and/or alkali hydroxide and an alkaline earth and/or alkali hypohalide.

Discovered by Hofmann at the end of the nineteenth century, this reaction is used to convert an amide function into a primary amine function with one carbon atom less. The detailed reaction mechanism is presented below.

In the presence of a BrØnsted base (e.g., soda), a proton is extracted from the amide.

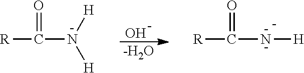

The amidate ion formed then reacts with the active chlorine (Cl₂) of the hypochlorite (e.g. NaClO which is in equilibrium: 2 NaOH+Cl₂⇔NaClO+NaCl+H₂O) to produce an N-chloramide. The BrØnsted base (e.g. NaOH) extracts a proton from the chloramide to form an anion. The anion loses a chloride ion to form a nitrene which undergoes isocyanate rearrangement.

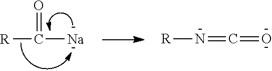

By reaction between the hydroxide ion and the isocyanate, a carbamate is formed.

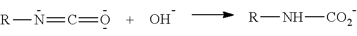

After decarboxylation (removal of CO₂) from the carbamate, a primary amine is obtained.

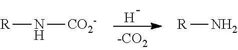

For the conversion of all or part of the amide functions of a (co)polymer comprising an amide group into an amine function, two main factors are involved (expressed in molar ratios). These are:
Alpha=(alkali and/or alkaline earth hypohalide/amide group) and
Beta=(alkali and/or alkaline earth hydroxide/alkali and/or alkaline earth hypohalide).

The polymer according to the invention can also be obtained by carrying out a glyoxalation reaction on a polymer obtained by polymerization of at least one monomer obtained by the method according to the invention or of at least one monomer as previously described in the “Monomer” section, said polymer comprising, with the glyoxalation reaction, at least one monomer unit advantageously of acrylamide or methacrylamide. More specifically, the glyoxalation reaction involves a reaction of at least one aldehyde on the polymer, thus allowing said polymer to be functionalized. Advantageously, the aldehyde may be chosen from the group comprising glyoxal, glutaraldehyde, furan dialdehyde, 2-hydroxyadipaldehyde, succinaldehyde, starch dialdehyde, 2.2 dimethoxyethanol, diepoxy compounds, and combinations thereof. Preferably, the aldehyde compound is glyoxal.

According to the invention, the polymer may be in liquid, gel or solid form when its preparation includes a drying step such as spray drying, drum drying, radiation drying, such as microwave drying, or fluid bed drying.

According to the invention, the water-soluble polymer preferably has a molecular weight between 1000 and 40 million g/mol. The polymer may be a dispersant, in which case its molecular weight is preferably between 1000 and 50,000 g/mol. The polymer may have a higher molecular weight, typically between 1 and 30 million g/mol. The molecular weight is understood as weight average molecular weight. The polymer according to the invention may also be a superabsorbent capable of absorbing from 10 to 500 times its weight in water.

The molecular weight is advantageously determined by the intrinsic viscosity of the (co)polymer. The intrinsic viscosity can be measured by methods known to the person skilled in the art and can be calculated from the reduced viscosity values for different (co)polymer concentrations by a graphical method entailing plotting the reduced viscosity values (y-axis) against the concentration (x-axis) and extrapolating the curve down to zero concentration. The intrinsic viscosity value is plotted on the y-axis or using the least squares method. The molecular weight can then be determined using the Mark-Houwink equation:

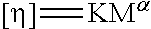

[η] represents the intrinsic viscosity of the (co)polymer determined by the solution viscosity measurement method.
K represents an empirical constant.
M represents the molecular weight of the (co)polymer.
α represents the Mark-Houwink coefficient.
K and a depend on the specific (co)polymer-solvent system.

The co-monomers combined with the monomer according to the invention to obtain the polymer of the invention, are preferably at least partially, or more preferably totally renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention, and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention; and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil;
at least 1 mol %, preferably between 5 mol % and 90 mol %, more preferably between 10 mol % and 80 mol % of at least one third monomer comprising an ethylenic unsaturation, said third monomer being different from the first and the second monomers, and being at least partially renewable and non-fossil.

The polymer according to the invention may comprise four or more different monomers.

In a preferred embodiment, the second and the possible other monomers have a bio-sourced carbon content ranging between 5 wt % and 100 wt %, preferably 10 wt % and 100 wt %, relative to the total carbon weight in the related monomer, the bio-sourced carbon content being measured according to ASTM D6866-21 Method B.

In this preferred embodiment, the second and the possible other monomers are preferably chosen from acrylamide, methacrylamide, (meth)acrylic acid and/or one of the salts, an oligomer of acrylic acid, 2-acrylamido-2-methylpropane sulfonic acid (ATBS) and/or a salt thereof, N-vinylformamide (NVF), N-vinylpyrrolidone (NVP), dimethyldiallylammonium chloride (DADMAC), a substituted acrylamide having the formula CH₂═CHCO—NR¹R², R¹ and R² being, independently of each other, a linear or branched carbon chain CₙH₂ₙ₊₁, wherein n is between 1 and 10.

In the whole invention, it will be understood that the molar percentage of the monomers (excluding any cross-linking agents) of the polymer is equal to 100%.

Preferably, the polymer according to the invention comprises a bio-sourced carbon content ranging between 5 wt % and 100 wt % relative to the total carbon weight in said polymer, the bio-sourced carbon content being measured according to ASTM D6866-21 Method B.

The dimethylaminoethanol, and/or the formula (II) compound, and/or the alkylating agent may be non-segregated, partially segregated, or totally segregated. The preferences developed in the method section apply to this section describing the polymer.

In a particular embodiment, the dimethylaminoethanol, and/or the formula (II) compound, and/or the alkylating agent may be partially or totally recycled. The preferences developed in the method section apply to this section describing the polymer.

The invention equally relates to a polymer obtained according to a method comprising the following steps:
Recycling at least one material that is at least partially renewable and non-fossil to obtain dimethylaminoethanol, and/or the formula (II) compound;
Reacting the dimethylaminoethanol and the formula (II) compound thus obtained in order to obtain the formula (I) compound, preferably according to a biological method conducted in the presence of a biocatalyst comprising a hydrolase enzyme; Polymerize said formula (I) monomer thus obtained, optionally with at least one second monomer comprising at least an ethylenic unsaturation to obtain a polymer.

The invention also relates to the use of at least one monomer obtained by the method according to the invention in order to synthesize a polymer.

### Using the Polymer According to the Invention

The invention also relates to the use of the polymer according to the invention in the recovery of hydrocarbons (oil and/or gas); in drilling and cementing of wells; in the stimulation of hydrocarbon wells (oil and/or gas), for example hydraulic fracturing, conformation, diversion; in the treatment of water in open, closed or semi-closed circuits; in the treatment of fermentation slurry, treatment of sludge; in paper manufacturing; in construction; in wood processing; in hydraulic composition processing (concrete, cement, mortar and aggregates); in the mining industry; in the formulation of cosmetic products; in the formulation of detergents; in textile manufacturing; in battery component manufacturing; in geothermal energy; in sanitary napkin manufacturing; or in agriculture.

The invention also relates to the use of the polymer according to the invention as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant.

### Method Using the Polymer According to the Invention

The present invention also relates to the various methods described hereinafter, wherein the polymers of the invention are used to improve application performance.

The invention also relates to a method for enhanced oil and/or gas recovery by sweeping a subterranean formation comprising the following steps:
a. Preparing an injection fluid from a polymer according to the invention with water or brine,
b. Injecting the injection fluid into a subterranean formation,
c. Sweeping the subterranean formation with the injected fluid,
d. Recovering an aqueous mixture of oil and/or gas.

The invention also relates to a method for hydraulic fracturing of subterranean oil and/or gas reservoirs comprising the following steps:
a. Preparing an injection fluid from a polymer according to the invention, with water or brine, and with at least one proppant,
b. Injecting said fluid into the subterranean reservoir and fracturing at least a portion thereof to recover oil and/or gas.

In the methods describe hereinabove, the polymer is preferably a high molecular weight polymer (greater than 8 million daltons). It is preferably linear. It is preferably in the form of a powder, an inverse emulsion, a partially dehydrated inverse emulsion, or in the form of a “clear”, i.e. a dispersion of solid polymer particles in an aqueous or oily fluid. The powder form is preferably obtained by gel or spray drying of an inverse emulsion. It also involves a composition comprising an inverse emulsion of a polymer according to the invention and solid particles of a polymer according to the invention.

The invention also relates to a method of stimulation of a subterranean formation comprising the following steps:
a. Preparing an injection fluid from a polymer according to the invention with water or brine,
b. Injecting the injection fluid into a subterranean formation,
c. Partially or totally plugging the subterranean formation with the injected fluid, said plugging being temporary or permanent.

The invention also related a method of drilling and/or cementing a well in a subterranean formation comprising the following steps:
a. Preparing an injection fluid from a polymer according to the invention with water or brine,
b. Injecting said drilling and/or cementing fluid into the subterranean formation via the drill head in at least one step of drilling or cementing a well.

Drilling and cementing a well are two successive steps in creating a well in a subterranean formation. The first step is drilling with the drilling fluid, while the second step is cementing the well with the cementing fluid. The invention also relates to a method of injecting an intermediate fluid (“spacer fluid”) injected between the drilling fluid and the cementing fluid, said intermediate fluid comprising at least one polymer according to the invention. This intermediate fluid prevents contamination between the cementing fluid and the drilling fluid.

When drilling and cementing a well, the polymer according to the invention can be used as a fluid loss additive in well cement compositions in order to reduce fluid loss from the cement compositions to permeable formations or zones into or through which the cement compositions are pumped. In primary cementing, loss of fluid, i.e., water, to permeable formations or subterranean zones can lead to premature gelling of the cement composition, so that bridging the annular space between the permeable formation or zone and the drill string cemented therein prevents the cement composition from being placed along the entire length of the ring.

The invention also relates to a method of inerting clays in hydraulic compositions for construction purposes, said method comprising a step of adding to the hydraulic composition or one of its constituents at least one clay inerting agent, characterized in that the clay inerting agent is a polymer according to the invention.

Clays can absorb water and cause poor performance of building materials. When the polymer of the invention is used as a clay inhibitor, it allows in particular to avoid the clay swelling which may cause cracks thus weakening any building.

The hydraulic composition may be a concrete, cement, mortar or aggregate. The polymer is added to the hydraulic composition or to one of its constituents advantageously at a dosage of 2 to 200 ppm of inerting agent relative to the weight of aggregate.

In this method of inerting clays, clays include, but are not limited to, 2:1 swelling clays (such as smectite), or 1:1 swelling clays (such as kaolin) or 2:1:1 swelling clays (such as chlorite). The term “clay” generally refers to magnesium and/or aluminum silicate, including phyllo silicates with a lamellar structure. However, in the present invention, the term “clay” also includes clays having no such structure, such as amorphous clays.

The invention also relates to a method for manufacturing a sheet of paper, cardboard or the like, whereby, before a sheet is formed, a step is performed entailing adding to a suspension of fibers, at one or more injection points, at least one polymer according to the invention. The polymer may provide dry strength or retention properties or wet strength. It may also improve paper formation, drainage and dewatering capabilities.

The method can be used successfully to manufacture packaging papers and cardboards, coating papers, sanitary and household papers, any type of paper, cardboard or the like.

The post-modified polymers described in the “Polymers” section, in particular the post-modified polymers by Hoffman reaction or by glyoxalation reaction, are particularly advantageous in methods for manufacturing paper, cardboard or the like.

Retention properties are understood to mean the capability to retain the suspended materials of the paper pulp (fibers, fines, fillers (calcium carbonate, titanium oxide), . . . ) on the forming fabric, thus in the fibrous mat that will make up the final sheet. The mode of action of the retention agents is based on the flocculation of these suspended materials in water. Indeed, the flocs formed are more easily retained on the forming sheet.

The retention of fillers involves retaining specifically the fillers (small mineral species with little affinity with cellulose). Substantial improvement of retention of fillers leads to a clarification of white water by retaining the fillers in the sheet and by increasing its grammage. It also gives the possibility to replace part of the fibers (the most expensive species in the composition of paper, cardboard or similar) with fillers (lower costs) in order to reduce manufacturing costs.

As concerns dewatering (or drainage) properties, it is the capacity of the fibrous mat to evacuate or drain the maximum amount of water so that the sheet dries as quickly as possible, in particular during manufacturing of the sheet.

These two properties (retention and drainage) being intricately linked, one depending on the other, the issue is therefore to find the best compromise between retention and drainage. Generally, the person skilled in the art refers to a retention and drainage agent because these are the same types of products used to improve these two properties.

Fibrous suspension is understood to mean thick pulp or diluted pulp which are composed of water and cellulose fibers. The thick stock, with a dry matter concentration of more than 1% or even more than 3%, is located upstream of the fan pump. The thin stock, with a dry mass concentration of generally less than 1%, is located downstream of the fan pump.

The polymer can be added to the thick stock or to the thin stock. It can be added at the level of the fan pump or the headbox. Preferably, the polymer is added before the headbox.

In the method for making paper, cardboard or the like according to the invention, the polymer according to the invention may be used alone or in combination with a secondary retention agent. Preferably, a secondary retention agent selected from organic polymers and/or inorganic microparticles is added to the fiber suspension.

This secondary retention agent added to the fibrous suspension is advantageously chosen from anionic polymers in the broad sense, which can therefore be (without being limiting) linear, branched, cross-linked, hydrophobic, associative and/or inorganic microparticles (such as bentonite, colloidal silica).

The invention also relates to a method for treating a suspension of solid particles in water resulting from mining or oil sands operations, comprising contacting said suspension with at least one polymer according to the invention. Such a method can be carried out in a thickener, which is a holding zone, generally in the form of a tube section of several meters in diameter with a conical bottom wherein the particles can settle. According to a specific embodiment, the aqueous suspension is transported by means of a pipe to a thickener, and the polymer is added to said pipe.

According to another embodiment, the polymer is added to a thickener that already contains the suspension to be treated. In a typical mineral processing operation, the suspensions are often concentrated in a thickener. This results in a higher density sludge that exits the bottom of the thickener, and an aqueous fluid released from the treated suspension (called liquor) that exits by overflow, from the top of the thickener. Generally, the addition of the polymer increases the concentration of the sludge and increases the clarity of the liquor.

According to another embodiment, the polymer is added to the particulate suspension during transport of said suspension to a deposition area. Preferably, the polymer is added in the pipe that conveys said suspension to a deposition zone. It is on this deposition area that the treated suspension is spread in preparation for dewatering and solidification. The deposition areas can be either open, such as an unconfined area of soil, or enclosed, such as a basin, cell.

An example of such treatments during transport of the suspension is spreading the suspension treated with the polymer according to the invention on the soil in preparation for dewatering and solidification and then spreading a second layer of treated suspension on top of the solidified first layer. Another example is the continuous spreading of the suspension treated with the polymer according to the invention in such a way that the treated suspension falls continuously on the suspension previously discharged in the deposition area, thus forming a mass of treated material from which water is extracted.

According to another embodiment, the water-soluble polymer is added to the suspension and a mechanical treatment is performed, such as centrifugation, pressing or filtration.

The water-soluble polymer can be added simultaneously in different stages of the suspension treatment, i.e., for example, in the pipe carrying the suspension to a thickener and in the sludge exiting the thickener which will be conveyed either to a deposition area or to a mechanical treatment device.

The invention also relates to a method for treating municipal or industrial water, comprising the introduction into said water to be treated of at least one polymer according to the invention. Effective water treatment requires the removal of dissolved compounds, and dispersed and suspended solids from the water. Generally, this treatment is enhanced by chemicals such as coagulants and flocculants. These are usually added to the water stream ahead of the separation unit, such as flotation and sedimentation.

The polymers according to the invention can be advantageously used to coagulate or flocculate suspended particles in municipal or industrial wastewater. Generally, they are used in combination with inorganic coagulants such as alum.

They can also be used advantageously to treat the sludge produced from the treatment of this wastewater. Sewage sludge (be it urban or industrial) is the main waste produced by a treatment plant from liquid effluents. Generally, sludge treatment involves dewatering it. This dewatering can be performed by centrifugation, filter press, belt press, electro-dewatering, sludge drying reed beds, solar drying. It is used to decrease sludge water concentration.

In this municipal or industrial water treatment process, the polymer according to the invention is preferably linear or branched. It is preferably in the form of a powder, an inverse emulsion or a partially dehydrated inverse emulsion. The powder form is preferably obtained by gel or spray drying from an inverse emulsion.

The invention also relates to an additive for a cosmetic, dermatological or pharmaceutical composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to cosmetic, dermatological or pharmaceutical compositions comprising at least one polymer according to the invention.

In particular, reference may be made to application FR2979821 on behalf of L'OREAL for the manufacture of such compositions and description of the other ingredients of such compositions. The said compositions may be in the form of a milk, a lotion, a gel, a cream, a gel cream, a soap, a bubble bath, a balm, a shampoo or a conditioner. The use of said compositions for the cosmetic or dermatological treatment of keratinous materials, such as the skin, scalp, eyelashes, eyebrows, nails, hair and/or mucous membranes is also an integral part of the invention. Such use comprises application of the composition to the keratinous materials, possibly followed by rinsing with water.

The invention also relates to an additive for detergent composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to detergent compositions for household or industrial use comprising at least one polymer according to the invention. In particular, reference may be made to the applicant's application WO2016020622 for the manufacture of such compositions and description of the other ingredients of such compositions.

“Detergent compositions for household or industrial use” are understood to mean compositions for cleaning various surfaces, particularly textile fibers, hard surfaces of any kind such as dishes, floors, windows, wood, metal or composite surfaces. Such compositions include, for example, detergents for washing clothes manually or in a washing machine, products for cleaning dishes manually or for dishwashers, detergent products for washing house interiors such as kitchen elements, toilets, furnishings, floors, windows, and other cleaning products for universal use.

The polymer used as an additive, e.g., thickener, for a cosmetic, dermatological, pharmaceutical, or detergent composition is preferably cross-linked. It is preferably in the form of a powder, an inverse emulsion or a partially dehydrated inverse emulsion. The powder form is preferably obtained by spray drying from an inverse emulsion.

The invention equally relates to a thickener for pigment composition used in textile printing, said thickener comprising at least one polymer according to the invention. The invention also relates to a textile fiber sizing agent, said agent comprising at least one polymer according to the invention.

The invention also relates to a process for manufacturing superabsorbent from the monomer according to the invention, a superabsorbent obtained from at least one monomer according to the invention, said superabsorbent to be used for absorbing and retaining water in agricultural applications or for absorbing aqueous liquids in sanitary napkins. For example, the superabsorbent agent is a polymer according to the invention.

The invention also relates to a method for manufacturing sanitary napkins wherein a polymer according to the invention is used, for example as a superabsorbent agent.

The invention also relates to the use of the polymer according to the invention as a battery binder. The invention also relates to a battery binder composition comprising the polymer according to the invention, an electrode material and a solvent. The invention also relates to a method for manufacturing a battery comprising making a gel comprising at least one polymer according to the invention and filling same into said battery. Mention may be made of lithium ion batteries which are used in a variety of products, including medical devices, electric cars, aircraft and, most importantly, consumer products such as laptops, cell phones and cameras.

Generally, lithium ion batteries (LIBs) include an anode, a cathode, and an electrolyte material such as an organic solvent containing a lithium salt. More specifically, the anode and cathode (collectively, the “electrodes”) are formed by mixing an electrode active material (anode or cathode) with a binder and solvent to form a paste or sludge that is then applied and dried onto a current collector, such as aluminum or copper, to form a film on the current collector. The anode and cathode are then stacked and wound before being housed in a pressurized case containing an electrolyte material, all of which together form a lithium-ion battery.

In a lithium battery, the binder plays several important roles in both mechanical and electrochemical performance. Firstly, it helps disperse the other components in the solvent during the manufacturing process (some also act as a thickener), thus allowing for even distribution. Secondly, it holds the various components together, including the active components, any conductive additives, and the current collector, ensuring that all of these parts stay in contact. Through chemical or physical interactions, the binder connects these separate components, holding them together and ensuring the mechanical integrity of the electrode without a material impact on electronic or ionic conductivity. Thirdly, it often serves as an interface between the electrode and the electrolyte. In this role, it can protect the electrode from corrosion or the electrolyte from depletion while facilitating ion transfer across this interface.

Another important point is that the binders must have a certain degree of flexibility so that they do not crack or develop defects. Brittleness can create problems during manufacturing or assembly of the battery.

Given all the roles it plays in an electrode (and in the battery as a whole), choosing a binder is critical in ensuring good battery performance.

The invention also relates to a method for manufacturing sanitary napkins wherein a polymer according to the invention is used, for example as a superabsorbent agent.

As previously described, the circular economy is an economic system devoted to efficiency and sustainability that minimizes waste by optimizing value generated by resources. It relies heavily on a variety of conservation and recycling practices in order to break away from the current more linear “take-make-dispose” approach.

Therefore, with material recycling being a major and growing concern, recycling processes are developing rapidly and enabling the production of materials that can be used to produce new compounds or objects. Recycling materials does not depend on the origin of the material and as long as it can be recycled, it is considered as a technical progress. Although the origin of the material to be recycled may be renewable and non-fossil, it may also be fossil.

### Specific Objects are Described Hereinafter.

A first particular object relates to a method for obtaining a formula (I) monomer comprising the reaction between a formula (II) compound and dimethylaminoethanol, R₂ being a hydrogen atom or a CH₃ group, R₃ being a hydrogen atom or an alkyl group comprising from 1 to 8 carbon atoms, characterized in that the dimethylaminoethanol is obtained at least partially, preferentially totally, from a recycling process of a renewal and non-fossil material, or a fossil material. This method may comprise a step of salification or quaternization (using an alkylating agent alkylant) of the formula (I) monomer.

The dimethylaminoethanol, and/or the formula (II) compound, and/or the alkylating agent may be non-segregated, partially segregated, or totally segregated. The same preferences developed in the method section apply to this section of the description.

In a particular embodiment, the dimethylaminoethanol, and/or the formula (II) compound, and/or the alkylating agent may be partially or totally recycled. The same preferences developed in the method section apply to this section of the description.

A second particular object relates to a formula (I) monomer obtained by reacting a formula (II) compound, R₂ being a hydrogen atom or a CH₃ group, R₃ being a hydrogen atom or an alkyl group comprising from 1 to 8 carbon atoms, with dimethylaminoethanol, preferentially by a biological method conducted in the presence of a biocatalyst comprising a hydrolase enzyme, said dimethylaminoethanol obtained at least partially, preferentially totally from a recycling process of a renewable and non-fossil material, or a fossil material, and/or, preferentially and, said formula (II) compound obtained at least partially, preferentially totally from a recycling process of a renewable and non-fossil material, or a fossil material.

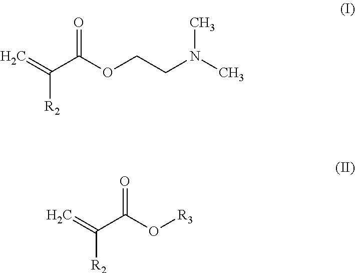

A third particular object relates to dimethylaminoethyl (meth)acrylate obtained by reacting methyl (meth)acrylate with dimethylaminoethanol, preferably by a biological method conducted in the presence of a biocatalyst comprising a hydrolase enzyme, said dimethylaminoethanol and/or said methyl (meth)acrylate obtained at least partially, preferentially totally from a recycling method of a renewable and non-fossil material, or a fossil material.

The invention further relates to the salified or quaternized form of dimethylaminoethyl (meth)acrylate. It may be quaternized with an alkylating agent, preferentially with an alkyl halide, e.g. methyl chloride, or dialkyl sulfate, e.g. dimethyl sulfate, diethyl sulfate, or benzyl chloride. The preferred alkylating agent is methyl chloride.

A fourth particular object relates to a polymer obtained by polymerization of at least one formula (I) monomer as just previously described.

A fifth specific object relates to the use of a polymer obtained by polymerization of at least one formula (I) monomer as just previously described, in the oil and/or gas recovery, in drilling and cementing of wells; in the stimulation of oil and/or gas wells (for example hydraulic fracturing, conformation, diversion), in the treatment of water in open, closed or semi-closed circuits, in the treatment of fermentation slurry, treatment of sludge, in paper manufacturing, in construction, in wood processing, in hydraulic composition processing (concrete, cement, mortar and aggregates), in the mining industry, in the formulation of cosmetic products, in the formulation of detergents, in textile manufacturing, in battery component manufacturing; in geothermal energy; in sanitary napkin manufacturing; or in agriculture.

A sixth specific object relates to the use of a polymer obtained by polymerization of at least one formula (I) monomer as just previously described as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant.

A seventh specific object relates to a polymer obtained according to a method comprising the following steps:
Recycling at least one material that is at least partially renewable and non-fossil, or a fossil

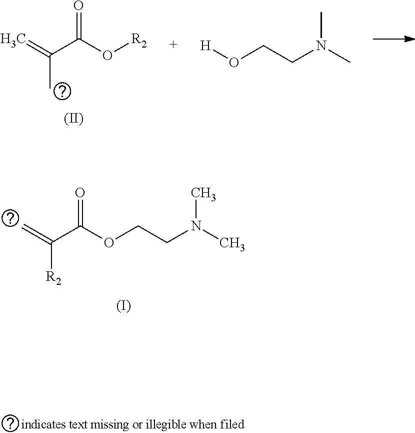

material, to obtain dimethylaminoethanol, and/or the formula (II) compound;
Reacting the dimethylaminoethanol and the formula (II) compound thus obtained in order to obtain the formula (I) compound, preferably according to a biological method conducted in the presence of a biocatalyst comprising a hydrolase enzyme;
Polymerize said formula (I) monomer thus obtained, optionally with at least one second monomer comprising at least an ethylenic unsaturation to obtain a polymer.

Said dimethylaminoethanol and/or formula (II) compound being preferentially totally “segregated”, i.e. derived from a separate pipeline and treated separately.

In an alternative embodiment, they are partially “segregated” and partially “non-segregated”. In this case, the weight ratio between the “segregated” part and the “non-segregated” part is preferentially between 99:1 and 25:75, preferably between 99:1 and 50:50. In an alternative embodiment, it is totally “segregated”.

### FIGURES

FIGS. 1 through 4 are graphs showing percent friction reduction versus time for each of the polymers.

### EXAMPLES

The following examples are related to synthesising a compound (I) according to the invention. This is to illustrate in a clear and non-limiting manner the advantages of the invention.

In the examples below:
Compound (I) is dimethylaminoethyl acrylate, annotated ADAME.
Compound (II) is methyl acrylate.
Dimethylaminoethanol is annotated DMOH.

### Description of the Purity Test

The purity of dimethylaminoethyl acrylate (or ADAME) is determined by gas phase chromatography, according to the following conditions:
DB-WAX UI column, 60 m×0.32 mm ID, 50 μm film
Temperature of injector: 250° C.
Oven: 80° C. for 5 minutes, followed by a ramp of 4° C./min up to 125° C. for 2 minutes, followed by a ramp of 35° C./min up to 240° C.
Temperature of detector: 250° C.
Injection volume: 2 μL in split ratio 1:200 with saver gas at 20 m/min after 5 minutes
Detector: FID (AUTOSYSTEM XL type from Perkin Elmer)
Detection gas: 30 mL/min in H₂ and 400 mL/min in air
Carrier gas (He): 1.5 mL/min

Thanks to the use of external standards and by measuring the areas of the various impurity peaks, the purity of ADAME can be calculated.

I. Synthesis of Bio-Sourced ADAME with DBTO Catalyst:

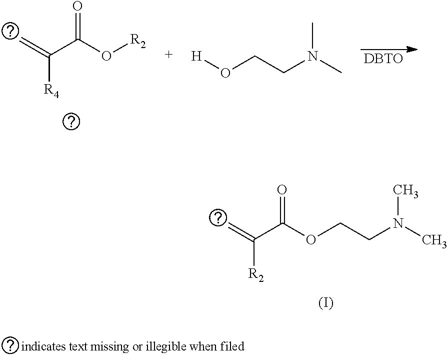

### Example 1: Synthesis of ADAME with Compound (II) of Fossil Origin

In this example, compound (II) is a methyl acrylate of fossil origin.

The origin of the DMOH will be either 100% fossil, or semi-fossil, or 100% of renewable and non-fossil origin.

For DMOH to form, two precursors are required: an ethanol precursor and a methanol precursor.

The DMOH of renewable and non-fossil origin can come from the treatment of residues from the paper pulp industry (“tall oil” in English), or from agricultural waste in order to form the bioethanol precursor (and therefore the bio oxide of ethylene). Methanol, on the other hand, can come from the treatment of municipal waste, biomass, by fermentation or recycling of carbon dioxide. Alternatively, the amino fraction of DMOH can also come from green ammonia. A DMOH of renewable and non-fossil origin, as described in the examples which follow, has precursors which are all of renewable and non-fossil origin.

The semi-fossil origin of DMOH, as described in the examples which follow, comes from the renewable and non-fossil origin of at least one of these precursors, when the other will have a fossil origin. It will be either the precursor bioethanol+methanol (source 1), or the precursor ethanol+biomethanol+green ammonia (source 2)

The fossil origin of DMOH comes from a fossil ethylene.

The level of ¹⁴C is measured according to the ASTM D6866-21 standard, method B. This standard makes it possible to characterize the bio-sourced nature of a chemical compound by determining the bio-sourced carbon level of said compound.

890 g of methyl acrylate (compound II), 460 g of DMOH, 130 g of hexane, 18 g of dibutyl tin oxide (DBTO) and 1 g of phenothiazine are added, with stirring, to a 2000 mL jacketed reactor.

Are then added to the above mixture, 90 g of hexane.

The mixture is heated using a heating unit supplying the jacket of the reactor, until a temperature of 80° C. is reached.

The temperature of the mixture is maintained at 80° C. for 7 hours.

The synthesis reaction is initiated once hexane and methanol vapors are condensed and collected; hexane is continuously introduced into the reaction medium in order to compensate for the quantity which is distilled.

After 7 hours of reaction, the reaction medium is sampled in order to be analysed by gas phase chromatography in order to determine the degree of conversion of the DMOH.

The reaction medium is distilled using a vacuum pump, under reduced pressure at a temperature of 95° C.

Three fractions are collected at different pressures, a first fraction of distillate is collected under a reduced pressure of 80 mbar absolute. A second fraction of distillate is collected under a reduced pressure of 6 mbar absolute. Finally, a last fraction of distillate is collected under a reduced pressure of 4 mbar absolute.

A test set is carried out according to the preceding protocol by adjusting the origin of the DMOH and its percentage in ¹⁴C (see table 2).

The wt % of ¹⁴C is indicative of the nature of the carbon. A “zero pMC” represents the total absence of measurable ¹⁴C in a material, thus indicating a fossil carbon source.

The vinyl ethanol (VOE) level is an indicator of the level of impurities transformed during the DMOH production process. The higher the rate, the more difficult the polymerization will be and the final application performance will be impacted.

To validate the DMOH to ADAME conversion test, the DMOH conversion rate must be greater than or equal to 93% coupled with an ADAME purity greater than or equal to 99.8% (see table 2).

TABLE 2

| | | | | | Quantity | Purity |
|---|---|---|---|---|---|---|
| | | | | | of | of the |
| | | VOE | wt % | % | ADAME | ADAME |
| | Origin of | rate | ¹⁴C of | conversion | obtained | obtained |
| | DMOH | (ppm) | DMOH | of DMOH | (g) | (%) |
| CEx 1 | Fossil | 6 | 0 | 90 | 1115 | 99.2 |
| CEx 2 | Fossil | 8 | 0 | 92 | 1120 | 99.3 |
| Inv 1 | semi-fossil | 0 | 40 | 93 | 1128 | 99.9 |
| | (Source 1) | | | | | |
| Inv 2 | semi-fossil | 1 | 50 | 97 | 1130 | 99.8 |
| | (Source 1) | | | | | |
| Inv 3 | semi-fossil | 4 | 40 | 94 | 1128 | 99.8 |
| | (Source 2) | | | | | |
| Inv 4 | semi-fossil | 1 | 50 | 95 | 1130 | 99.8 |
| | (Source 2) | | | | | |
| Inv 5 | Not fossil | 1 | 70 | 94 | 1127 | 99.8 |
| Inv 6 | Not fossil | 2 | 80 | 96 | 1131 | 99.9 |
| Inv | Not fossil | 2 | 100 | 98 | 1140 | 99.9 |

(CEx = counter-example; Inv = example according to the invention)

The Applicant observes that the DMOHs partially or totally of renewable and non-fossil origin make it possible to validate the conversion test.

### Example 2: Synthesis of ADAME with Compound (II) of Renewable and Non-Fossil Origin

In this example, compound II is a methyl acrylate of non-fossil origin containing 100% ¹⁴C.

The protocol previously described in example 1 is reproduced.

The conditions for validating the DMOH to ADAME conversion test are the same as in example 1.

TABLE 3

| | | | | | ADAME | ADAME |
|---|---|---|---|---|---|---|
| | | VOE | wt % | | quantity | purity |
| | Origin of | rate | of ¹⁴C of | conversion | obtained | obtained |
| | DMOH | (ppm) | DMOH | of DMOH | (g) | (%) |
| CEx 3 | Fossil | 6 | 0 | 91 | 1117 | 99.2 |
| CEx 4 | Fossil | 8 | 0 | 92 | 1120 | 99.3 |
| Inv 8 | semi-fossil | 0 | 40 | 94 | 1128 | 99.9 |
| | (Source 1) | | | | | |
| Inv 9 | semi-fossil | 1 | 50 | 97 | 1130 | 99.9 |
| | (Source 1) | | | | | |
| Inv 10 | semi-fossil | 4 | 40 | 97 | 1131 | 99.8 |
| | (Source 2) | | | | | |
| Inv 10 | semi-fossil | 1 | 50 | 95 | 1130 | 99.8 |
| | (Source 2) | | | | | |
| Inv 12 | Not fossil | 1 | 70 | 97 | 1132 | 99.9 |
| Inv 13 | Not fossil | 2 | 80 | 98 | 1138 | 99.9 |
| Inv 14 | Not fossil | 2 | 100 | 99 | 1142 | 99.9 |

(CEx = counter-example; Inv = example according to the invention)

The Applicant observes that the nature of compound (II) influences the validity of the conversion test.

II. Synthesis of ADAME with Lipase Type Biocatalyst:

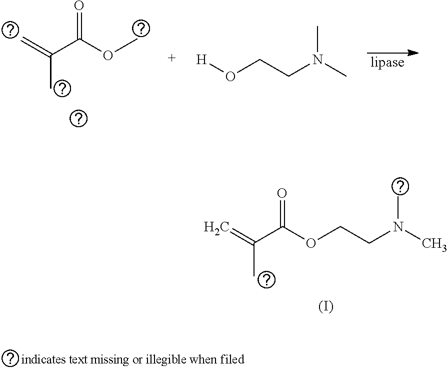

### Example 3: Synthesis of ADAME Entirely of Renewable and Non-Fossil Origin

2500 g of methyl acrylate, 300 g of DMOH, 650 g of hexane, 250 g of Lipase CalB (Novozyme company) and 5 g of MEHQ are added, with stirring, to a 5000 mL reactor having a double jacket.

450 g of hexane is added to the above mixture.

The mixture is heated by a heating unit supplying the reactor jacket until a temperature of 40° C. is reached. Once this temperature has been reached, the mixture will remain maintained for 30 hours at 40° C.

The synthesis reaction is initiated once hexane and methanol vapors are condensed and collected. Hexane is continuously introduced into the reaction medium in order to compensate for the quantity which is distilled.

After 30 hours at 40° C., the reaction medium is sampled to determine the degree of conversion of the DMOH.

The reaction medium is distilled using a vacuum pump under reduced pressure at a temperature of 95° C.

Three fractions are collected at different pressures, a first fraction of distillate is collected under a reduced pressure of 80 mbar absolute. A second fraction of distillate is collected under a reduced pressure of 6 mbar absolute. Finally, a last fraction of distillate is collected under a reduced pressure of 4 mbar absolute

As in the previous examples, different origins of DMOH will be tested.

To validate the DMOH to ADAME conversion test, the conversion rate must be greater than or equal to 80% and must be combined with a purity of ADAME greater than or equal to 99.8%. (see Table 4).

TABLE 4

| | | | | | ADAME | ADAME |
|---|---|---|---|---|---|---|
| | | VOE | wt % | % | quantity | purity |
| | DMOH | rate | of ¹⁴C of | conversion | obtained | obtained |
| | origin | (ppm) | DMOH | of DMOH | (g) | (%) |
| CEx 5 | Fossil | 6 | 0 | 65 | 310 | 99.2 |
| CEx 6 | Fossil | 8 | 0 | 70 | 335 | 99.3 |
| Inv 15 | semi-fossil | 0 | 40 | 84 | 400 | 99.9 |
| | (Source 1) | | | | | |
| Inv 16 | semi-fossil | 1 | 50 | 86 | 415 | 99.9 |
| | (Source 1) | | | | | |
| Inv 17 | semi-fossil | 4 | 40 | 86 | 415 | 99.8 |
| | (Source 2) | | | | | |
| Inv 18 | semi-fossil | 1 | 50 | 85 | 407 | 99.8 |
| | (Source 2) | | | | | |
| Inv 19 | Not fossil | 1 | 70 | 86 | 415 | 99.9 |
| Inv 20 | Not fossil | 2 | 80 | 88 | 422 | 99.9 |
| Inv 21 | Not fossil | 2 | 100 | 89 | 425 | 99.9 |

(CEx = counter-example; Inv = example according to the invention)

The bio-sourced nature of the precursors influences the conversion test as described above.

### Example 4: Quaternised Monomers According to the Invention

In a 1000 L stainless steel reactor, with a pressure-resistant jacket, 300 g of monomers from the previous examples are introduced with stirring. The reactor is closed and pressurized with 1 absolute bar of air.

The reaction medium is heated by a heating unit supplying the reactor jacket until a temperature of 40° C. is reached. The methyl chloride is introduced with a flow rate of 111 g/h. As soon as 10% of the stoichiometry of the methyl chloride is reached, water is introduced concomitantly at a flow rate of 42 g/h. When all the water has been introduced (i.e. 100 g), the introduction of methyl chloride is stopped, and the reactor is returned to atmospheric pressure.

Air is then bubbled through for 30 minutes in order to degas the excess methyl chloride.

An aqueous solution of ADAME quaternised with methyl chloride is thus obtained. The concentration of this salt is 80% in water.

A test set is carried out according to the preceding protocol by adjusting the origin of the ADAME, as well as the origin of the methyl chloride and its percentage of ¹⁴C (see table 5).

Methyl chloride of non-fossil origin can come from the treatment of residues from the paper pulp industry (“tall oil” in English), from agricultural waste or from the treatment of municipal waste, biomass, by fermentation or recycling of carbon dioxide. Alternatively, the chlorinated fraction of the methyl chloride can also be derived from chlorine or green hydrogen chloride, that is to say made from a renewable energy source.

The rate of ¹⁴C in the different products is measured according to the ASTM D6866-21 method B standard.

TABLE 5

| | | Origin of | wt % of ¹⁴C | wt % of ¹⁴C |
|---|---|---|---|---|
| | Origin of | methyl | methyl | ADAME quaternised |
| | ADAME | chloride | chloride | with methyl chloride |
| CEx 7 | CEx 1 | Fossil | 0 | 0 |
| CEx 8 | CEx 3 | Fossil | 0 | 0 |
| CEx 9 | CEx 5 | Fossil | 0 | 0 |
| M1 | Inv 7 | Fossil | 0 | 87.5 |
| M2 | Inv14 | Biomethanol | 80 | 97.5 |
| M3 | Inv 20 | Biomethanol | 100 | 82.5 |
| M4 | Inv 21 | Biomethanol | 100 | 100 |

(CEx = counter-example; Inv = example according to the invention)

### III. Polymer According to the Invention

### Example 5: Biodegradability Test of Polymers P1 to P5

In a 2000 mL beaker, are added deionized water and monomers (monomers from table 5)

The resulting solution is cooled to 5-10° C. and transferred to an adiabatic polymerisation reactor.

Nitrogen bubbling is carried out for 30 minutes in order to eliminate all traces of dissolved oxygen.

Are then added to the reactor:
0.45 g of 2,2′-azobisisobutyronitrile,
1.5 mL of an aqueous solution at 2.5 g/L of 2,2′-Azobis[2-(2-imidazolin-2-yl)propane]dihydrochloride,
1.5 mL of a 1 g/L aqueous solution of sodium hypophosphite,
1.5 mL of a 1 g/L aqueous solution of tert-butyl hydroperoxide,
1.5 mL of an aqueous solution at 1 g/L of ammonium sulphate and iron (II) hexahydrate (Mohr's salt).

After a few minutes, the nitrogen bubbling is stopped. The polymerization reaction then proceeds for 4 hours to reach a temperature peak. At the end of this time, the polymer gel obtained is chopped and dried, then again crushed and sieved to obtain a polymer in powder form.

The biodegradability (after 28 days) of the polymers obtained is evaluated according to the OECD 302B standard.

TABLE 6

| Polymer | P1 | P2 | P3 | P4 | P5 | CEx10 | CEx11 | CEx12 | CEx13 |
|---|---|---|---|---|---|---|---|---|---|
| Mass of quaternised | 202.5 | 202.5 | 202.5 | 202.5 | 202.5 | 202.5 | 202.5 | 202.5 | 202.5 |
| ADAME (g) | | | | | | | | | |
| Monomer | M1 | M2 | M3 | M4 | M4 | Cex7 | CEx8 | CEx9 | CEx9 |
| wt % ¹⁴C of monomer | 87.5 | 97.5 | 82.5 | 100 | 100 | 0 | 0 | 0 | 0 |
| Mass of acrylamide (g) | 276 | 276 | 276 | 276 | 276 | 276 | 276 | 276 | 276 |
| wt % ¹⁴C of acrylamide | 0 | 0 | 0 | 0 | 100 | 0 | 0 | 0 | 100 |
| Mass of water (g) | 522 | 522 | 522 | 522 | 522 | 522 | 522 | 522 | 522 |
| % biodegradability | 35 | 40 | 33 | 40 | 50 | 12 | 15 | 14 | 17 |

(CEx = counter-example)

The Applicant observes that the polymers according to the invention have a biodegradability profile up to 60% higher than the polymers not containing bio-sourced monomers as described in the invention.

### Example 6: Measurement of Insolubility Rate in Polymer Solutions

The UL viscosity (Brookfield viscosity), insolubility velocity and insolubility point are measured on a polymer composed of 70 mol % acrylamide and 30 mol % quaternised ADAME prepared by conventional bulk polymerisation.

UL viscosity is measured using a Brookfield viscometer fitted with a UL adapter, the unit of which rotates at 60 rpm (0.1 wt % of polymer in a saline solution of 1M sodium chloride) between 23 and 25° C.

The insolubility rate is measured by transferring Ig of the polymer solution into 200 mL of water at 20° C., stirring for 2 h, then the dissolved solution is filtered through a 4 cm diameter filter with a porosity of 200 μm. After complete draining of the filtered solution, the filter paper is weighted down. In the case of a non-filterable solution, the sieve filter is placed at 105° C. for 4 hours. The residual mass is used to determine the insoluble amount, the insolubility rate is related to the initial mass of the polymer. The vinyl acrylate impurity creates covalent bonds between the 2-dimethylaminoethyl acrylate monomers, resulting in aggregates that do not pass through the filter.

The insolubility point is the number and size of the aggregates on the filter. The following scale is used: point (pt) between 1 and 3 mm; large point (bp) for more than 3 mm (visual count).

TABLE 7

| Polymer | P1 | P2 | P3 | P4 | P5 | CEx10 | CEx11 | CEx12 | CEx13 |
|---|---|---|---|---|---|---|---|---|---|
| Viscosity UL (Cps) | 5.3 | 5.3 | 5.3 | 5.4 | 5.4 | 5.1 | 5.2 | 5.1 | 5.3 |
| Number of | 12 | 10 | 7 | 8 | 5 | 30 | 15 | 23 | 20 |
| insolubles (points) | | | | | | | | | |
| Rate of insolubles | 2 | 3 | 1 | 3 | 0 | 7 | 7 | 5 | 3 |

(CEx = counter-example)

The Applicant observes that the polymers which are entirely of renewable and non-fossil origin have fewer insolubles.

### IV. Use of the Polymer According to the Invention

### Example 7: Use of the Polymer as an Additive in a Papermaking Method

Retention agents are polymers added to cellulose fibre pulps prior to paper formation to increase the retention efficiency of the paper.

Type of pulp used: Virgin fibre pulp:

A wet pulp is obtained by disintegrating a dry pulp to obtain a final aqueous concentration of 1 wt %. It is a neutral pH pulp composed of 90% bleached virgin long fibres, 10% bleached virgin short fibres and 30 wt % additional GCC (ground calcium carbonate) (Hydrocal® 55 from Omya) based on fibre weight.

### Evaluation of Total Retention and Filler Retention

For all the following tests, the polymer solutions are prepared at 0.5 wt %. After 45 minutes of preparation, the polymer solutions are diluted 10 times before injection.

The different results are obtained using a Britt Jar device with a stirring speed of 1000 rpm.

The process sequence is as follows:
T=0 s: Stirring of 500 mL of paper pulp at a concentration of 0.5 wt %.
T=10 s: Addition of retention agent (300 g dry polymer/ton of dry pulp).
T=20 s: Removal of the first 20 mL representing the dead volume under the cloth, followed by collection of 100 mL of white water.

The first pass retention percentage (% FPR), corresponding to the total retention, is calculated according to the following formula: % FPR=(C_{HB}-C_{WW})/C_{HB}*100

The first pass ash retention percentage (% FPAR) is calculated using the following formula: % FPAR=(A_{HB}-A_{WW})/A_{HB}*100 with:
C_{HB}: Headbox consistency
C_{WW}: White Water Consistency
A_{HB}: Headbox ash consistency

For each of these analyses, the highest values represent the best performance.

### Evaluation of Gravity Drainage Performance Using the “Canadian Standard Freeness (CSF)”

In a beaker, the pulp is processed at a stirring speed of 1,000 rpm.

The process sequence is as follows:

−T=0 s: Stirring of 500 mL of paper pulp at a concentration of 0.6 wt %.
T=10 s: Addition of retention agent (300 g dry polymer/ton of dry pulp).
T=20 s: Stopping the stirring and adding the necessary amount of water to obtain 1 litre.

This litre of pulp is transferred to the Canadian Standard Freeness Tester and the TAPPI procedure T227om-99 is applied.

The volume, expressed in mL, gives a measure of gravitational freeness. The higher the value, the better the gravity drainage.

This performance can also be expressed by calculating the percent improvement relative to the blank (% CSF). The highest values represent the best performance.

The same polymers as above are tested and the results are presented below.

TABLE 8

| Polymer | P1 | P2 | P3 | P4 | P5 | CEx10 | CEx11 | CEx12 | CEx13 |
|---|---|---|---|---|---|---|---|---|---|
| % FPAR | 31.5 | 33.2 | 30.1 | 32.3 | 34.6 | 20.3 | 20.7 | 20.8 | 21 |
| % FPR | 72.3 | 74 | 75.6 | 77 | 78.5 | 64.2 | 64.8 | 65 | 65.4 |
| % CSF | 7.3 | 12.3 | 17.5 | 18.2 | 19.7 | 1.5 | 2 | 3.4 | 4.9 |

(CEx = counter-example)

The Applicant observes that the polymers of the invention offer better performance as a retention agent for paper. With regard to drainage, a polymer prepared with only monomers according to the invention improves performance by more than 25%.

### Example 8: Measurement of Friction Reduction

Polymers P1 to P5 and CEx 10 to 13 are dissolved under agitation at a concentration of 10,000 ppm in a brine composed of water, 85 g of sodium chloride (NaCl) and 33.1 g of calcium chloride (CaCl₂, 2 H₂O) per litre of brine.

The resulting polymer salt solutions are then injected at a concentration of 0.5 pptg (part per thousand grams) into the circulating brine for the Flow Loop tests.

Indeed, to evaluate the friction reduction of each of the polymers and those from counterexamples 1 to 4, the reservoir of the loop of the Flow Loop (calibrated tube length (loop): 6 mn internal diameter of the tube: 4 mm) was filled 20 L of brine as described above.

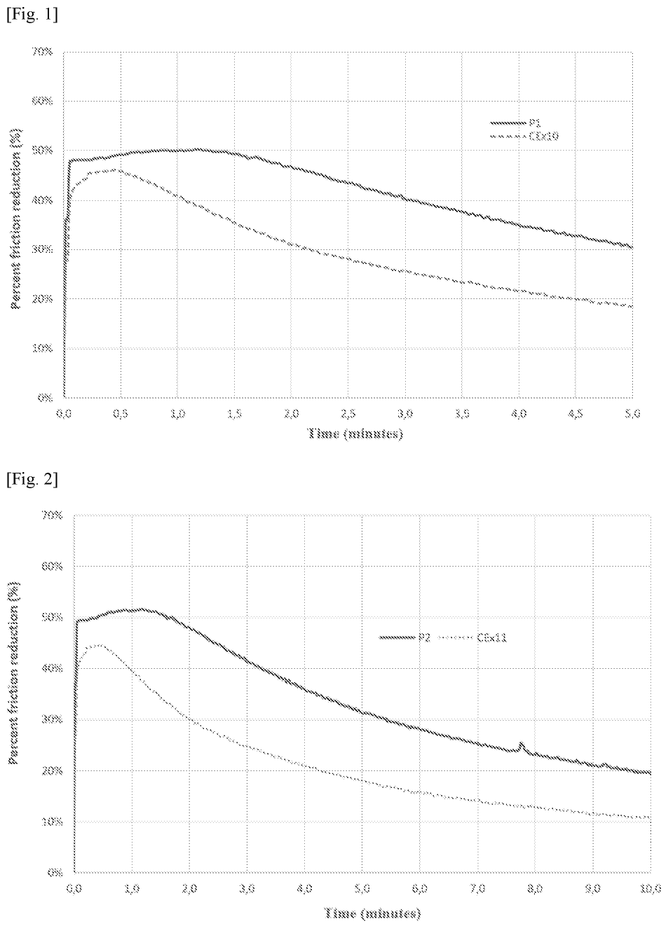

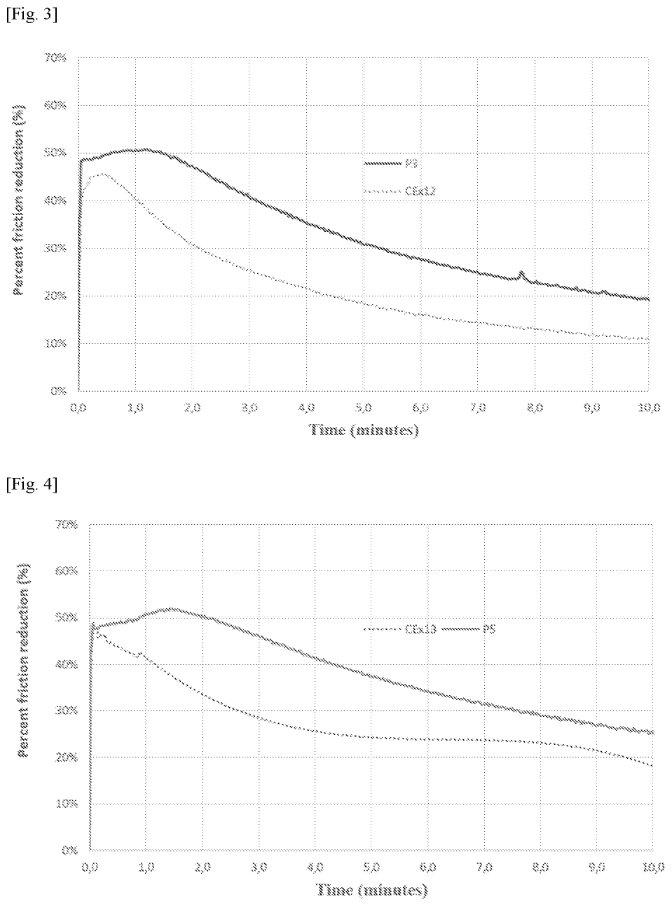

The brine is then circulated through the Flow Loop at a rate of 24 gallons/min. The polymer is added at a concentration of 0.5 pptg to the recirculating brine. The percentage of friction reduction is determined by measuring the pressure changes within the Flow Loop. FIGS. 1 through 4 are graphs showing percent friction reduction versus time for each of the polymers. These figures show that the injection fluids according to the invention allow an improved friction reduction.

1. A method for obtaining a formula (I) monomer comprising the reaction between a compound of formula (II) and dimethylaminoethanol,
R₂ being a hydrogen atom or a CH₃ group,
R₃ being a hydrogen atom or an alkyl group comprising from 1 to 8 carbon atoms, wherein the dimethylaminoethanol is at least partially renewable and non-fossil

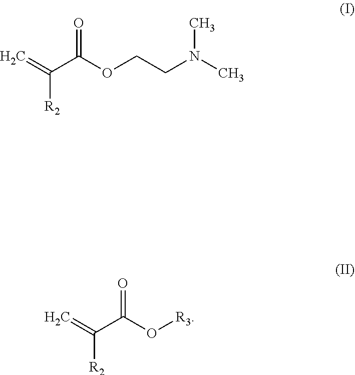

2. The method according to claim 1, wherein the dimethylaminoethanol has a bio-sourced carbon content of between 5 wt % and 100 wt % relative to the total carbon weight in said dimethylaminoethanol, the bio-sourced carbon content being measured according to the standard ASTM D6866-21 Method B.

3. The method according to claim 1, wherein the formula (II) compound has a bio-sourced carbon content of between 25 wt % and 100 wt % relative to the total carbon weight in the formula (II) compound, the bio-sourced carbon content being measured according to the standard ASTM D6866-21 Method B.

4. The method according to claim 1, wherein the formula (I) monomer has a bio-sourced carbon content of between 45 wt % and 100 wt % relative to the total carbon weight in said monomer, the bio-sourced carbon content being measured according to the standard ASTM D6866-21 Method B.

5. The method according to claim 1, wherein the formula (I) monomer is either salified or quaternized with an alkylating agent.

6. The method according to claim 6, wherein the alkylating agent has a bio-sourced carbon content of between 50 wt % and 100 wt % relative to the total carbon weight in said alkylating agent, the bio-sourced carbon content being measured according to the standard ASTM D6866-21 Method B.

7. The method according to claim 1, wherein the method is a biological method conducted in the presence of a biocatalyst comprising a hydrolase enzyme selected from lipases, esterases, glycosylases, and proteases; the hydrolase enzyme being in free form or immobilized on a substrate.

8. The method according to claim 7, wherein the enzyme is a lipase synthesized by a microorganism selected from the group consisting of Alcaligenes sp., Aspergillus sp., Mucor sp., Penicillium sp., Geotrichum sp., Rhizopus sp., Burkholderia sp., Candida sp., Pseudomonas sp., Thermomyces sp., and Candida Antarctica.

9. The method according to claim 1, wherein the dimethylaminoethanol and/or the formula (II) compound are partially or totally segregated.

10. The method according to claim 1, wherein the dimethylaminoethanol and/or the formula (II) compound are partially or totally derived by a recycling method.

11. A formula (I) monomer with a bio-sourced carbon content ranging between 45 wt % and 100 wt % relative to the total carbon weight in said monomer, the bio-sourced carbon content being measured according to ASTM D6866-21 Method B,
R₂ being a hydrogen atom or a CH₃ group

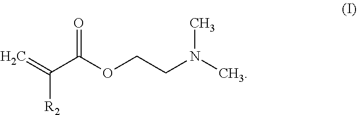

12. The formula (I) monomer wherein said monomer is obtained by reacting a formula (II) compound, R₂ being a hydrogen atom or a CH₃ group, R₃ being a hydrogen atom or an alkyl group comprising from 1 to 8 carbon atoms, with dimethylaminoethanol, preferably by a biological method carried out in the presence of a biocatalyst comprising a hydrolase enzyme,
and in that said dimethylaminoethanol having a bio-sourced carbon content of between 5 wt % and 100 wt % based on the total weight of carbon in said dimethylaminoethanol, and/or, preferably, said compound of formula (II) having a bio-sourced carbon content of between 5 wt % and 100 wt % based on the total weight of carbon in said compound of formula (II), the bio-sourced carbon content being measured according to ASTM D6866-21 Method B

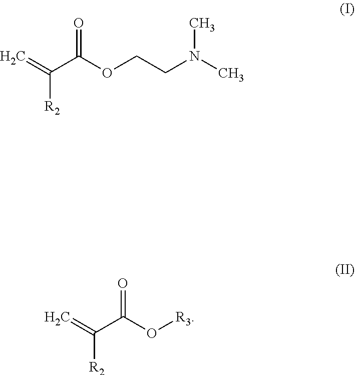

13. A bio-sourced dimethylaminoethyl (meth)acrylate with a bio-sourced carbon content ranging between 45 wt % and 100 wt % relative to the total carbon weight in said bio-sourced dimethylaminoethyl (meth)acrylate, the bio-sourced carbon content being measured according to ASTM D6866-21 Method B.

14. The bio-sourced dimethylaminoethyl (meth)acrylate, said bio-sourced dimethylaminoethyl (meth)acrylate obtained by reacting methyl (meth)acrylate with a dimethylaminoethanol, preferentially by a biological method carried out in the presence of a biocatalyst comprising a hydrolase enzyme,
and said dimethylaminoethanol having a bio-sourced carbon content ranging between 5 wt % and 100 wt % relative to the total carbon weight in said dimethylaminoethanol, and/or, preferentially and, said methyl (meth)acrylate having a bio-sourced carbon content ranging between 5 wt % and 100 wt % relative to the total carbon weight in said methyl (meth)acrylate, the bio-sourced carbon content being measured in accordance with ASTM D6866-21 Method B.

15. A salified or quaternized version of the bio-sourced dimethylaminoethyl (meth)acrylate according to claim 11.

16. A polymer obtained by polymerization of at least one monomer obtained according to the method according to claim 1, wherein the polymer is a copolymer of:
at least a first monomer obtained by a method according to claim 1, and
at least a second monomer different from the first monomer, said second monomer selected from the group consisting of nonionic monomers, anionic monomers, cationic monomers, zwitterionic monomers, monomers comprising a hydrophobic moiety, and mixtures thereof.

17. (canceled)

18. The polymer according to claim 16, wherein the polymer is a copolymer of:
at least 5 mol %, preferentially between 20 mol % and 90 mol %, more preferentially between 30 mol % and 99 mol % of a first monomer, said monomer being a monomer obtained by the method according to claim 1, and
at least 1 mol %, preferentially between 5 mol % and 95 mol %, more preferentially between 10 mol % and 80 mol % of at least one second monomer comprising an ethylenic unsaturation, said second monomer being different from the first monomer, and comprising a bio-sourced carbon content of between 5 wt % and 100 wt % relative to the total carbon weight in said second monomer, the bio-sourced carbon content being measured according to the standard ASTM D6866-21 Method B.

19. The polymer according to claim 18, wherein the second monomer is selected from acrylamide, (meth)acrylic acid and/or a salt thereof, an oligomer of acrylic acid, ATBS and/or a salt thereof, N-vinylformamide (NVF) N-vinylpyrrolidone (NVP), dimethyldiallylammonium chloride (DADMAC), or a substituted acrylamide of the formula CH₂═CHCO—NR¹R², R¹ and R² being, independently of each other, a linear or branched carbon chain CₙH₂ₙ₊₁, wherein n ranges between 1 and 10.

20. The polymer according to claim 16 comprising a bio-sourced carbon content ranging between 5 wt % and 100 wt % relative to the total carbon weight in said polymer, the bio-sourced carbon content being measured according to ASTM D6866-21 Method B.

21. (canceled)

22. (canceled)

23. (canceled)

24. (canceled)

25. A method for hydraulic fracturing of subterranean oil and/or gas reservoirs, comprising:
a. preparing an injection fluid from a polymer, according to claim 16, with water or brine, and with at least one proppant,
b. injecting said fluid into the subterranean reservoir and fracturing at least a portion thereof to recover oil and/or gas.

26. (canceled)

27. A method for making a sheet of paper or a cardboard, whereby, before forming said sheet, at least one polymer is added to a fiber suspension at one or more injection points according to claim 16.

28. A method for treating municipal and industrial water comprising adding into said municipal or industrial water at least one polymer according to claim 16.

29. (canceled)

30. (canceled)

31. (canceled)
